(19) United States
(12) Patent Application Publication
(10) Pub. No.: US 2017/0268031 A1
(43) Pub. Date: Sep. 21, 2017

(54) METHODS FOR BACTERIOPHAGE DETECTION

(71) Applicant: The Regents of the University of California, Oakland, CA (US)

(72) Inventors: Nitin NITIN, Vacaville, CA (US); Min WANG, Santa Monica, CA (US)

(73) Assignee: The Regents of the University of California, Oakland, CA (US)

(21) Appl. No.: 15/500,013

(22) PCT Filed: Jul. 21, 2015
(86) PCT No.: PCT/US15/41400

§ 371 Date: Jan. 27, 2017

Related U.S. Application Data
(60) Provisional application No. 62/031,091, filed on Jul. 30, 2014.

(51) Int. Cl. C12Q 1/04; B01L 7/00; G01N 33/04; B01L 3/00

(52) U.S. Cl. CPC C12Q 1/04; B01L 3/502715; B01L 7/00; B01L 3/502784; G01N 33/04; G01N 2333/245; G01N 2333/01; B01L 2300/0672; B01L 2300/0654; B01L 2200/0673; B01L 2300/18

(57) ABSTRACT

Provided are methods and devices for the detection of bacteriophages.

### CROSS-REFERENCE TO RELATED APPLICATIONS

This application is the U.S. national phase under 35 U.S.C. §371 of Intl. Appl. No. PCT/US2015/041400, filed Jul. 21, 2015, which claims the benefit under 35 U.S.C. §119(e) of U.S. Provisional Application No. 62/031,091, filed on Jul. 30, 2014, which are hereby incorporated herein by reference in their entireties for all purposes.

### STATEMENT OF GOVERNMENTAL SUPPORT

This invention was made with government support under Grant No. 2011-67021-20034 awarded by the United States Department of Agriculture—Agriculture and Food Research Initiative (USDA-AFRI). The government has certain rights in the invention.

### FIELD

Provided are methods and devices for the detection of bacteriophages.

### BACKGROUND

Bacteriophage (phage) contamination is one of the major causes of fermentation failure in the dairy (1, 2) and biochemical processing industries (3). The presence of phages, especially lytic phages (4) in the starter culture or raw material such as milk can slow the fermentation process (5), reduce lactic acid production (4) and affect quality of the end-product (4). It has been reported that phage contamination is the primary cause of economic loss in the dairy industry (6, 7), affecting up to 10% of all milk fermentation processes (7). To minimize phage contamination, the fermentation industry has implemented several strategies to control phages, including improved sanitation protocols (2, 5), rotation of starter culture (2, 5), and the development of phage-resistant bacterial starter strains (8, 9).

Despite careful control of phages, phage contamination cannot be completely eliminated (2). Therefore, early detection of phages in the starter culture itself or in the raw material is needed. Currently, phage contamination is detected by sampling the whey using traditional microbiological methods such as indicator tests (2, 5) and plaque assays (5, 9). However, these traditional assays often require an extended period of incubation (a few hours to days) before phage contamination could be detected (2). More recently, molecular based techniques such as polymerase chain reaction (PCR) (1, 10) and flow cytometry (11) have been developed to detect phages in starter culture. The reported detection limits for PCR and flow cytometry were between 10⁴-10⁷ PFU/mL (1, 2, 4), 10 and 10⁵ PFU/mL (11), respectively. One of the major drawbacks of PCR is that the technique cannot distinguish between viable and non-viable phages (2), and only known species of phages can be detected or quantified (2, 11). On the other hand, the flow cytometric method by Michelsen, et. al. quantified the number of membrane-compromised bacteria as an indication of phage infection (11). Using the side scatter plots, Michelsen, et. al. determined that the late stage phage infected cells have 30-50% decrease in scattered light compared to the control (11). However, flow cytometry is an expensive technique that requires a highly trained personnel to operate the instrument, gate the events and analyze the data (4); thus, it may not be a suitable method to detect phages in a typical fermentation industry.

### SUMMARY

In one aspect, provided is a method of assaying for bacteriophage in a sample suspected of comprising bacteriophage. In varying embodiments, the methods comprise:

a) creating a water-in-oil (W/O) emulsion, comprising:
i) suspending a bacterial cell mixture in an inner aqueous phase (W1) comprising a water soluble emulsifier and a cell viability dye, wherein the bacterial cell mixture comprises the sample suspected of comprising bacteriophage; and
ii) suspending droplets of the inner aqueous phase (W1) into an oil phase (O) comprising an oil and a hydrophobic emulsifier having an HLB value of 4 or less, thereby yielding a water-in-oil (W1/O) emulsion; and

b) detecting the cell viability dye, wherein detectable cell viability dye provides a signal when bacterial cells within the water-in-oil (W1/O) emulsion are non-viable, thereby indicating the presence of bacteriophage in the sample suspected of comprising bacteriophage. In varying embodiments, the method further comprises before step a) i) the step of mixing a sample suspected of comprising bacteriophage with a population of bacterial cells, thereby yielding a bacterial cell mixture.

In a related aspect, provided is a method of assaying for bacteriophage in a sample suspected of comprising bacteriophage. In varying embodiments, the methods comprise:

a) creating a water-in-oil-in-water (W1/O/W2) emulsion, comprising:
i) suspending a bacterial cell mixture in an inner aqueous phase (W1) comprising a water soluble emulsifier and a cell viability dye, wherein the bacterial cell mixture comprises the sample suspected of comprising bacteriophage;
ii) suspending droplets of the inner aqueous phase (W1) into an oil phase (O) comprising an oil and a hydrophobic emulsifier having an HLB value of 4 or less, thereby yielding a water-in-oil (W1/O) emulsion; and
iii) mixing the water-in-oil (W1/O) emulsion in an outer aqueous phase (W2), comprising at least one water soluble emulsifier having a hydrophilic lipophilic balance (HLB) value of 7 or greater, thereby creating a water-in-oil-in-water (W1/O/W2) emulsion comprising bacterial cells; and

b) detecting the cell viability dye, wherein detectable cell viability dye provides a signal when bacterial cells within the water-in-oil-in-water (W1/O/W2) emulsion are non-viable, thereby indicating the presence of bacteriophage in the sample suspected of comprising bacteriophage. In varying embodiments, the method further comprises before step a) i) the step of mixing a sample suspected of comprising bacteriophage with a population of bacterial cells, thereby yielding a bacterial cell mixture.

In another aspect, provided is a method of assaying for bacteria strains that are resistant to bacteriophage lysis, comprising:

a) creating a water-in-oil (W/O) emulsion, comprising:
i) suspending a bacterial cell mixture in an inner aqueous phase (W1) comprising a water soluble emulsifier and a cell viability dye; and
ii) suspending droplets of the inner aqueous phase (W1) into an oil phase (O) comprising an oil and a hydrophobic emulsifier having an HLB value of 4 or less, thereby yielding a water-in-oil (W1/O) emulsion; and

b) detecting the cell viability dye, wherein detectable cell viability dye provides a signal when bacterial cells within the water-in-oil (W1/O) emulsion are non-viable, thereby indicating the presence of bacteria susceptible to bacteriophage in the bacterial cell culture or mixture; and wherein nondetectable cell viability dye indicates the presence of bacteria resistant to bacteriophage in the bacterial cell culture or mixture.

In a further aspect, provided is a method of assaying for bacteria strains that are resistant to bacteriophage lysis, comprising:

a) creating a water-in-oil-in-water (W1/O/W2) emulsion, comprising:
i) suspending a bacterial cell mixture in an inner aqueous phase (W1) comprising a water soluble emulsifier and a cell viability dye;
ii) suspending droplets of the inner aqueous phase (W1) into an oil phase (O) comprising an oil and a hydrophobic emulsifier having an HLB value of 4 or less, thereby yielding a water-in-oil (W1/O) emulsion; and
iii) mixing the water-in-oil (W1/O) emulsion in an outer aqueous phase (W2), comprising at least one water soluble emulsifier having a hydrophilic lipophilic balance (HLB) value of 7 or greater, thereby creating a water-in-oil-in-water (W1/O/W2) emulsion comprising bacterial cells; and

b) detecting the cell viability dye, wherein detectable cell viability dye provides a signal when bacterial cells within the water-in-oil (W/O) emulsion are non-viable, thereby indicating the presence of bacteria susceptible to bacteriophage in the bacterial cell culture or mixture; and wherein nondetectable cell viability dye indicates the presence of bacteria resistant to bacteriophage in the bacterial cell culture or mixture.

With respect to embodiments of the methods, in varying embodiments, the detecting step comprises performing visual inspection. In varying embodiments, the method detects bacteriophage with a sensitivity of about 10⁴ PFU/mL or less by visual inspection. In varying embodiments, the detecting step comprises performing optical microscopy. In varying embodiments, the detecting step comprises performing optical flow cytometry. In varying embodiments, the method detects bacteriophage with a sensitivity of about 10² PFU/mL or less by optical microscopy or flow cytometry. In varying embodiments, the detecting step does not comprise performing one or more of flow cytometry, impedance spectroscopy or nucleic acid amplification. In varying embodiments, the method can be performed in 2 or fewer hours, e.g., in less than 120, 90, 60, 45, 30 minutes. In varying embodiments, the water soluble or hydrophilic emulsifier in the inner aqueous phase (W₁) has a hydrophilic lipophilic balance (HLB) value of 10 or greater. In varying embodiments, the emulsifier with a hydrophilic lipophilic balance (HLB) value of 10 or greater comprises a protein-based or proteinaceous emulsifier, e.g., whey protein isolate (WPI), soy protein isolate, caseins and/or milk proteins. In varying embodiments, the hydrophilic emulsifier in the inner aqueous phase (W₁) comprises a particle-based emulsifier. In varying embodiments, the cell viability dye is a fluorophore. In varying embodiments, the cell viability dye binds to or intercalates into DNA. In varying embodiments, the cell viability dye is selected from the group consisting of propidium iodide (PI), 7-aminoactinomycin D (7-AAD), DRAQ7™, and TO-PRO®-3 Iodide. In varying embodiments, the cell viability dye is selected from propidium iodide (PI), hexidium iodide, a carbocyanine, rhodamine 123, tetra methyl rhodamine, dialkylaminophenylpolyenylpyridinium, aminonaphthylethenylpyridinium, resazurin, formazan, red-fluorescent ethidium homodimer-1, calcein, tetrasodium (6E,6′E)-6,6-[(3,3′-dimethylbiphenyl-4,4′-diyl)di(1E)hydrazin-2-yl-1-ylidene]bis(4-amino-5-oxo-5,6-dihydronaphthalene-1,3-disulfonate) (Evans blue), (3Z,3′Z)-3,3′-[(3,3′-dimethylbiphenyl-4,4′-diyl)di(1Z)hydrazin-2-yl-1-ylidene]bis(5-amino-4-oxo-3,4-dihydronaphthalene-2,7-disulfonic acid) (Trypan blue), 7-aminoactinomycin D (7-AAD), DRAQ7™, eFluor® 455UV, eFluor® 450, eFluor® 506, eFluor® 520, eFluor® 660, eFluor® 780, Zombie Aqua™, Zombie Green™, Zombie NIR™, Zombie Red™, Zombie Violet™, Zombie UV™, and Zombie Yellow™. In varying embodiments, the cell viability dye is a colorimetric dye. In varying embodiments, the one or more bacteriophages are lytic bacteriophages. In varying embodiments, the one or more bacteriophages are lysogenic or temperate bacteriophages. In varying embodiments, the methods further comprise prior to the detecting step, inducing the lytic cycle of the lysogenic or temperate bacteriophages. In varying embodiments, the one or more bacteriophages are a member of a viral family selected from the group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, and Tectiviridae. In varying embodiments, the one or more bacteriophages are lytic to a bacterial cell selected from the group consisting of Campylobacter, Cronobacter, Escherichia, Salmonella, Lactococcus, Vibrio, Erwinia, Xanthomonas, Shigella, Staphylococcus, Streptococcus, Clostridium, Pseudomonas, Mycobacterium, Neisseria, and Bacilli. In varying embodiments, the bacteriophages are selected from the group consisting of lactococcal phage species (936, c2, c6A, 1483, T187, P087, 1358, KSY1, 949, and P335 phage species), T4 phage, T7 phage, phage A1511, phage Felix-Ol1, phage PHL 4, phage P7, ECML-4, ECML-117, ECML-134, phage A511, phage P100, ATCC accession no. PTA-5372, ATCC accession no. PTA-5373, ATCC accession no. PTA-5374, ATCC accession no. PTA-5375, ATCC accession no. PTA-5376, ATCC accession no. PTA-5377, phage FO1-E2, phage CJ6, phage Φ88, phage Φ35, NgoΦ6 and NgoΦ7, lambdoid prophages, phage β, Lambda phages, Mu-1, lactococcal lysogenic phages (φLC3, Tuc2009, bIL285, bIL286 and bIL309, bIL170, biL167), Lysogenic phages of S. aureus (8325-4, Ps6, 655, 248, W-26, U9, 655C, Oh-SO, 608, N-135, C-72), and mixtures thereof. In varying embodiments, the oil in the oil phase is liquid at room temperature, e.g., at 25-30° C. In varying embodiments, the oil in the oil phase is selected from mineral oil, canola oil, olive oil, corn oil, sunflower oil, safflower oil, peanut oil, coconut oil and fluorinated oils (e.g., perfluorodecalin). In varying embodiments, the hydrophobic emulsifier with an HLB value of 4 or less comprises a polyglycerol ester of fatty acid. In varying embodiments, the hydrophobic emulsifier with an HLB value of 4 or less comprises polyglycerol polyricinoleate (PGPR). In varying embodiments, the outer aqueous phase (W2) comprises a mixture comprising a bile salt, a zwitterionic detergent and a nonionic detergent. In varying embodiments, the outer aqueous phase (W2) comprises one or more bile salts, lecithin and Tween 20. In varying embodiments, the detecting step is performed in comparison to a control comprising the inner aqueous phase without bacteriophage.

In another aspect, provided is a portable device. In some embodiments, the portable device comprises a tubing in fluid communication from the upstream to downstream direction, with (i) a fluidic droplet generator, (ii) an incubator and (iii) a detector. In varying embodiments:

a) the upstream end of the tubing is in fluid communication with a sample reservoir;

b) the tubing within the fluidic droplet generator comprises a first upstream syringe comprising a needle comprising a beveled tip, wherein the beveled tip is pierced into the inner space of the tubing, wherein the inner space of the first syringe comprises an oil phase comprising an emulsifier; and a second downstream syringe comprising a needle comprising a beveled tip, wherein the beveled tip is pierced into the inner space of the tubing downstream from the needle of the first syringe, wherein the inner space of the second syringe comprises an aqueous phase comprising at least one detergent;

c) the incubator can hold a preselected or predetermined temperature in the range of about 4° C. to about 50° C.; and

d) the detector can detect a fluorescent or colorimetric signal. In some embodiments, one or more of the sample reservoir, the first syringe and the second syringe automatically deliver fluid. In some embodiments, the device weighs less than 10 kg, e.g., less than about 9 kg, 8 kg, 7 kg, 6 kg, 5 kg, 4 kg, 3 kg, 2 kg, 1 kg, or less. In some embodiments, the device has a desk or table footprint of less than about 200 in², e.g., less than about 190 in², 180 in², 170 in², 160 in², 150 in², 140 in², 130 in², 120 in², 110 in², 100 in², or less. In some embodiments, the inner space of the tubing has a diameter in the range of about 1/32 (0.03125) inches to about 1/16 (0.0625) inches. In some embodiments, the needle of the first syringe and/or the second syringe has a gauge from about 18 G to about 34 G, e.g., from about 25 G to about 30 G, e.g., 25 G, 26 G, 27 G, 28 G, 29 G, 30 G, 31 G, 32 G, 33 G or 34 G. In some embodiments, the portable device is as depicted in FIGS. 8, 9 and/or 10. In varying embodiments, the fluidic droplet generator is as described in FIG. 9. The portable device can be used to generate water-in-oil (W/O) droplets and/or water-in-oil-in-water (W/O/W) droplets.

In a further aspect, provided is a microfluidic device for creating water-in-oil (W/O) and/or water-in-oil-in-water (W1/O/W2) emulsion droplets. In varying embodiments the microfluidic device comprises one or more units or modules of channels comprising:
i) a first inlet in fluid communication with a first lumen or channel, the first inlet and first lumen or channel comprising an inner aqueous phase;
ii) a second inlet in fluid communication with a second lumen or channel, the second inlet and second lumen or channel comprising an oil phase, wherein the second lumen or channel is in fluid communication with the first lumen or channel;
iii) a third inlet in fluid communication with a third lumen or channel, the third inlet and third lumen or channel comprising an outer aqueous phase, wherein the third lumen or channel is in fluid communication with the first lumen or channel, wherein the third lumen or channel connects with the first lumen or channel downstream of where the second lumen or channel connects with the first lumen or channel; and
iv) an outlet for collecting water-in-oil (W/O) and/or water-in-oil-in-water (W1/O/W2) emulsion droplets, wherein the outlet is in fluid communication with the first lumen or channel. In some embodiments, the inner diameters of the first, second and third lumens or channels are from about 30 μm to about 150 μm. In some embodiments, the device is as depicted in FIGS. 11-12.

In a related aspect, provided is a microfluidic device for creating water-in-oil (W₁/O) emulsion droplets, comprising:
i) a first inlet in fluid communication with a first lumen, the first inlet and first lumen comprising an inner aqueous phase;
ii) a second inlet in fluid communication with a second lumen, the second inlet and second lumen comprising an oil phase, wherein the second lumen is in fluid communication with the first lumen; and
iii) an outlet for collecting water-in-oil (W₁/O) emulsion droplets, wherein the outlet is in fluid communication with the first lumen. In some embodiments, the inner diameters of the first, second and third lumens or channels are from about 30 μm to about 150 μm.

### BRIEF DESCRIPTION OF THE DRAWINGS

FIGS. 1A-D illustrate encapsulation of bacteria and phages in a W1/O/W2 emulsion microdroplet. A 5 mL disposable syringe that was filled with the O phase and fitted with a piece of Tygon PVC tubing was placed on a syringe pump, with the open end inserted into a microcentrifuge tube. The W1 phase was filled in the 1 mL disposable syringe fitted with a 27 G hypodermic needle, and was pierced into the Tygon tubing (Inset A). A constant O phase flow rate and W1 injection rate was maintained to continuously generate stable W1/O emulsion droplets (Inset B). The W1/O emulsion was collected in a microcentrifuge tube (Inset C). (D) To generate the W1/O/W2 emulsion microdroplets, an aliquot of the W1/O emulsion containing bacteria and phages was added to W2 phase in a second microcentrifuge tube using a cut pipet tip. The tube was subsequently shaken rigorously for a few seconds to form the emulsion microdroplets.

FIGS. 2A-C. (A) Schematic illustration of a W1/O/W2 emulsion microdroplet with the respective components in the W1, O and W2 phases. (B) Brightfield and (C) fluorescence microscopy image showing the close-up view of one W1/O/W2 emulsion microdroplet. The bacteria in the W1 phase were labeled with SYBR Green (green), and the O phase was labeled with BODIPY 665. The microscopy images were taken using an Olympus IX-71 inverted fluorescence microscope with a 10× objective (Olympus UPlanFLN).

FIGS. 3A-B illustrate size distribution of the W1/O/W2 emulsion microdroplets. (A) A representative brightfield image of the W1/O/W2 emulsion microdroplets taken using a 4× objective. (B) The size distribution of the W1/O/W2 emulsion microdroplets, n=1047.

FIGS. 4A-B illustrate fluorescence signal contrast between the encapsulated and non-encapsulated bacteria and phages samples. Representative fluorescence microscopy images of the (A) W/O/W encapsulated and (B) non-encapsulated samples containing 10⁸ CFU/mL of bacteria with 10⁶ PFU/mL of phages. The images were taken after 1 hr of incubation at 37° C. using an Olympus IX-71 inverted fluorescence microscope with a 4× objective.

FIGS. 5A-D. Qualitative analysis of phage amplification in the W1/O/W2 emulsion microdroplets. A membrane impermeable dye, PI was included in the W1 phase and the PI-labeled bacteria after one hour of incubation at 37° C. Representative normalized fluorescence microscopy images of the W1/O/W2 emulsion microdroplets with 10⁸ CFU/mL bacteria and (A) 0 PFU/mL (B) 10² PFU/mL (C) 10⁴ PFU/mL, and (D) 10⁶ PFU/mL at (i) t=0 hr and (ii) t=1 hr. The images were taken using an Olympus IX-7I inverted fluorescence microscope with a 4× objective.

FIG. 6 illustrates mean pixel intensity (MPI) inside the W1/O/W2 emulsion microdroplets. The MPI of the control, 10², 10⁴ and 10⁶ PFU/mL phages at t=0 hr (white bars) and after 1 hr of incubation at 37° C. (black bars). * p<0.001 within sample; # p<0.001 between samples. Mean±SE, n=170-407.

FIGS. 7A-B illustrate a co-axial microfluidic emulsion droplet generation setup.

FIGS. 8A-B illustrate the bacteriophage detection system comprised of a microfluidic generator, incubator and detector.

FIG. 9 illustrates detection methods using (A) flow cytometry and (B) optical imaging, to assess phage contamination inside the emulsion droplets.

FIG. 10 illustrates detection methods using (A) flow cytometry and (B) optical imaging, to assess phage contamination inside the emulsion droplets.

FIG. 11 illustrates a cross-flow microfluidic emulsion droplet generation setup.

FIGS. 12A-C illustrate a cross-flow microfluidic emulsion droplet generation setup.

### DETAILED DESCRIPTION

1. Introduction

Bacteriophage contamination of starter culture and raw material poses a major problem in the fermentation industry. Provided are methods for the rapid detection of phage contamination, e.g., in a sample suspected of containing bacteriophage contamination (e.g., a food product sample, e.g., a fermented food product, milk, whey, etc.), using water-in-oil-in-water (W/O/W) and/or water-in-oil (W/O) emulsion microdroplets. Model bacteria with varying concentrations of phages were encapsulated in W/O/W and/or W/O emulsion microdroplets using a simple needle-in-tube setup. The detection of phage contamination was accomplished in one hour using the propidium iodide labeling of the phage-infected bacteria inside the W/O/W emulsion microdroplets. Using this approach, a detection limit of 10² PFU/mL of phages was achieved quantitatively, while 10⁴ PFU/mL of phages could be detected qualitatively based on visual comparison of the fluorescence images.

Compartmentalization of biomolecules in discrete W/O/W and/or W/O emulsion microdroplets is an attractive approach for detection and screening, and has been used in applications such as PCR reactions (12-15), phage display amplification (16, 17), and quantification of enzyme activity (18) and protein expression (19) at the single cell level. While many studies have demonstrated that uniformly sized emulsion microdroplets can be produced rapidly using chip-based microfluidics (20-25), the commercialization this technology has been limited due to scale up cost (26), and handling of large sample volumes. Therefore, large-scale methods such as homogenization (27), rigorous stirring (28), and tubing-based setup (needle-in-tube) (29-33) are simpler and more practical approaches to the chip-based technology for industrial use.

Using the needle-in-tube setup, a model bacteria and phage were encapsulated in discrete W/O/W emulsion microdroplets. The presence of phages in each W/O/W emulsion was detected using fluorescence microscopy based on the propidium iodide (PI) labeling of dead bacteria. Since the PI dye can only bind to the DNA inside the bacteria with damaged membrane, a PI-labeled bacterium would be indicative of a phage infection. The high local concentration of bacteria and phages in discrete W/O/W emulsion microdroplets provided a good signal-to-noise ratio for quantification using fluorescence microscopy, and the presence of phages could be detected within one hour.

Given the simplicity and sensitivity of this approach, this method can be readily adapted to any strains of bacteria and phages that are commonly used for fermentation, e.g., using a portable device, and is applicable to rapid detection of phage contamination, e.g., in food products suspected of having bacteriophage contamination.

2. Methods of Bacteriophage Detection

Generally, the methods for bacteriophage detection comprise encapsulating a population of bacterial cells suspected of bacteriophage infection with a cell viability dye in a water-in-oil (W/O) and/or water-in-oil-in-water (W/O/W) emulsion microdroplet. Non-viable bacterial cells, e.g., which have been infected with a bacteriophage, will be stained with the cell viability dye, indicating the presence of bacteriophage contamination. The methods can be performed rapidly, with bacteriophage contamination being detected within 2 or fewer hours, e.g., 120, 90, 60, 45, 30 or fewer minutes, and without requiring performing flow cytometry, impedance spectroscopy or nucleic acid amplification. Detection performed employing visual inspection can achieve a sensitivity of 10⁴ PFU/mL of phages. Detection performed employing optical microscopy, or optionally flow cytometry can achieve a sensitivity of 10² PFU/mL of phages.

a. Suspending the Bacterial Cells in an Inner Aqueous Phase (W1)

The methods provide for the rapid detection of phage contamination, e.g., in a sample suspected of containing bacteriophage contamination (e.g., a food product sample, e.g., a fermented food product, milk, whey, etc.), using water-in-oil-in-water (W/O/W) and/or water-in-oil (W/O) emulsion microdroplets. In varying embodiments, the sample may already be a mixture containing a population of bacterial cells. Such samples can be used directly in the methods, as long as the bacterial cells in the sample are susceptible to lysis in the presence of bacteriophage. In varying embodiments, the sample suspected of containing bacteriophage contamination does not contain bacteria. In embodiments testing samples which do not contain bacteria, a population of bacteria susceptible to lysis in the presence of bacteriophage can be mixed into the sample, thereby creating a bacterial cell mixture.

A population of bacterial cells, e.g., from a bacterial cell culture or mixture suspected of containing or being monitored for the presence of bacteriophage contamination is suspended in an inner aqueous phase (W1) comprising a water soluble emulsifier with a hydrophilic lipophilic balance (HLB) value of 10 or greater and a cell viability dye. Generally, the methods are performed on a scale such that the volume of the inner aqueous phase fits within a microtube, e.g., less than about 2 mls, e.g., less than about 2.0, 1.8, 1.5, 1.2, 1.0, 0.8 mLs. In varying embodiments, the inner aqueous phase comprises about 1-10%, e.g., about 5% (w/v) surfactant (e.g., water soluble emulsifier, e.g., with a hydrophilic lipophilic balance (HLB) value of 10 or greater).

With respect to the emulsifier in the inner aqueous phase (W1), any water soluble emulsifier can be used. In varying embodiments, the emulsifier in the inner aqueous phase (W1) has a hydrophilic lipophilic balance (HLB) value of 10 or higher. In varying embodiments, the emulsifier comprises a protein-based or proteinaceous emulsifier, e.g., whey protein isolate (WPI), soy protein isolate, caseins and/or milk proteins. Other emulsifiers that can find use in the inner aqueous phase include without limitation, e.g., PEG-7 Glyceryl Cocoate (HLB=10); PEG-20 Almond Glycerides (HLB=10); Linoleamide DEA (HLB=10); PEG-25 Hydrogenated Castor Oil (HLB=10.8); Stearamide MEA (HLB=11); Glyceryl Stearate and/or PEG-100 Stearate (HLB=11); Polysorbate 85 (HLB=11); PEG-7 Olivate (HLB=11); Cetearyl Glucoside (HLB=11); PEG-8 Oleate (HLB=11.6); Polyglyceryl-3 Methyglucose Distearate=12 Oleth-10 (HLB=12.4); Oleth-10/Polyoxyl 10 Oleyl Ether NF (HLB=12.4); Ceteth-10 (HLB=12.9); PEG-8 Laurate (HLB=13); Cocamide MEA (HLB=13.5); Polysorbate 60 NF (HLB=14.9); Polysorbate 60 (HLB=14.9); Polysorbate 80 (HLB=15); Isosteareth-20 (HLB=15); PEG-60 Almond Glycerides (HLB=15); Polysorbate 80 NF (HLB=15); PEG-20 Methyl Glucose Sesquistearate (HLB=15); Ceteareth-20 (HLB=15.2); Oleth-20 (HLB=15.3); Steareth-20 (HLB=15.3); Steareth-21 (HLB=15.5); Ceteth-20 (HLB=15.7); Isoceteth-20 (HLB=15.7); Polysorbate 20 (HLB=16.7); Polysorbate 20 NF (HLB=16.7); Laureth-23 (HLB=16.9); PEG-100 Stearate (HLB=18.8); Steareth-100 (HLB=18.8); PEG-80 Sorbitan Laurate (HLB=19.1); starch-based emulsifiers including starch particles; protein-based or proteinaceous emulsifiers including soy protein isolate, or casein; and hydrophilic particles including silica particles.

With respect to the cell viability dyes, both fluorophores and colorimetric dyes find use. In varying embodiments, the cell viability dye is a fluorophore. Illustrative fluorophores to distinguish live versus dead (e.g., non-infected versus infected with bacteriophage, respectively) bacterial cells include without limitation is selected from the group consisting of propidium iodide (PI), 7-aminoactinomycin D (7-AAD), DRAQ7™, and TO-PRO®-3 Iodide. In varying embodiments, the cell viability dye is selected from propidium iodide (PI), hexidium iodide, a carbocyanine, rhodamine 123, tetra methyl rhodamine, dialkylaminophenylpolyenylpyridinium, aminonaphthylethenylpyridinium, resazurin, formazan, red-fluorescent ethidium homodimer-1, calcein, tetrasodium (6E,6′E)-6,6-[(3,3′-dimethylbiphenyl-4,4′-diyl)di(1E)hydrazin-2-yl-1-ylidene]bis(4-amino-5-oxo-5,6-dihydronaphthalene-1,3-disulfonate) (Evans blue), (3Z,3′Z)-3,3′-[(3,3′-dimethylbiphenyl-4,4′-diyl)di(1Z)hydrazin-2-yl-1-ylidene]bis(5-amino-4-oxo-3,4-dihydronaphthalene-2,7-disulfonic acid) (Trypan blue), 7-aminoactinomycin D (7-AAD), DRAQ7™, eFluor® 455UV, eFluor® 450, eFluor® 506, eFluor® 520, eFluor® 660, eFluor® 780, Zombie Aqua™, Zombie Green™, Zombie NIR™, Zombie Red™, Zombie Violet™, Zombie UV™, and Zombie Yellow™. Zombie dyes are available from BioLegend, Inc. (on the internet at biolegend.com).

The methods can be used to detect the presence of any kind of bacteriophage contamination of a bacterial culture or mixture. The bacteriophage can be lytic or lysogenic (e.g., temperate). When detecting lysogenic or temperate bacteriophages, the methods can further entail the step of inducing the lytic cycle of the lysogenic bacteriophages. Induction of the lytic cycle is generally performed prior to the detection step, and can be performed after formation of the water-in-oil (W1/O) emulsion or water-in-oil-in-water (W1/O/W2) emulsion. In varying embodiments, this can be accomplished by exposing the bacterial cell mixture to an external stimulus that induces the lytic cycle. Such external stimuli are known in the art and include without limitation changes in temperature, UV light exposure, chemicals such as antibiotics or combination of these. Because the methods do not employ nucleic acid amplification, the methods do not rely on the knowledge of bacteriophage genomic sequences. In varying embodiments, the one or more bacteriophages subject to detection are a member of a viral family selected from the group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, and Tectiviridae. Illustrative bacteriophages that can be detected using the methods and devices described herein include without limitation, e.g., lactococcal phage species (936, c2, c6A, 1483, T187, P087, 1358, KSY1, 949, and P335 phage species), T4 phage, T7 phage, phage A1511, phage Felix-O1, phage PHL 4, phage P7, ECML-4, ECML-117, ECML-134, phage A511, phage P100, ATCC accession no. PTA-5372, ATCC accession no. PTA-5373, ATCC accession no. PTA-5374, ATCC accession no. PTA-5375, ATCC accession no. PTA-5376, ATCC accession no. PTA-5377, phage FO1-E2, phage CJ6, phage Φ88, phage Φ35, NgoΦ6 and NgoΦ7, lambdoid prophages, phage β, Lambda phages, Mu-1, lactococcal lysogenic phages (φLC3, Tuc2009, bIL285, bIL286 and bIL309, biL170, biL167), Lysogenic phages of S. aureus (8325-4, Ps6, 655, 248, W-26, U9, 655C, Oh-SO, 608, N-135, C-72), and mixtures thereof. See, e.g., Siddiqui et al., Applied Microbiology, January 1974, p. 278-280).

The methods can be used to detect bacteriophage (e.g., lytic or lysogenic) contamination of any bacterial culture or mixture. In varying embodiments, the one or more bacteriophages are lytic to or infect a bacterial cell selected from the group consisting of Campylobacter, Cronobacter, Escherichia, Salmonella, Lactococcus, Vibrio, Erwinia, Xanthomonas, Shigella, Staphylococcus, Streptococcus, Clostridium, Pseudomonas, Mycobacterium, Neisseria, and Bacilli.

b. Forming Water-in-Oil (W1/O) Emulsion

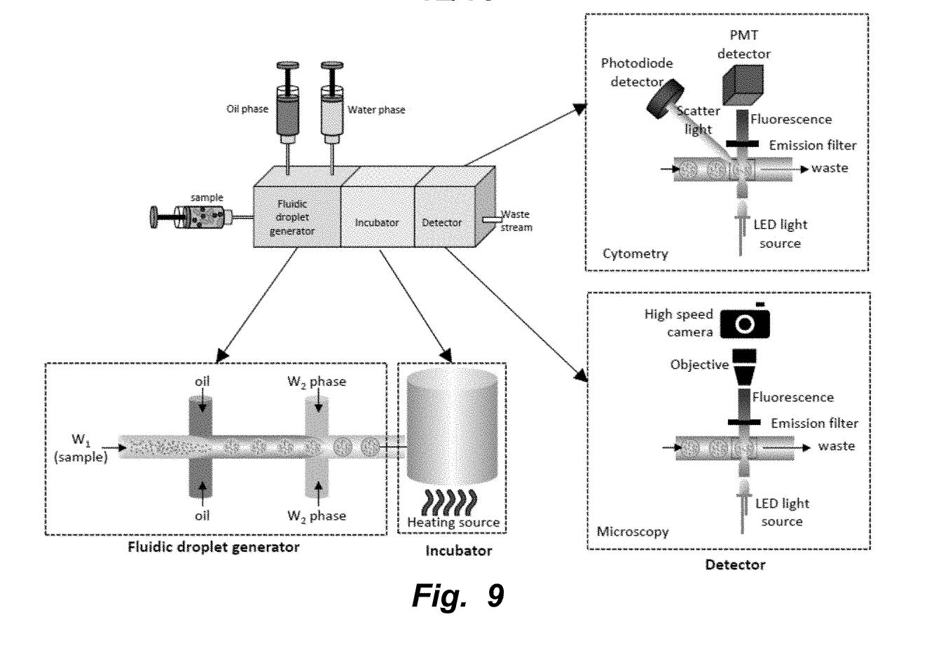

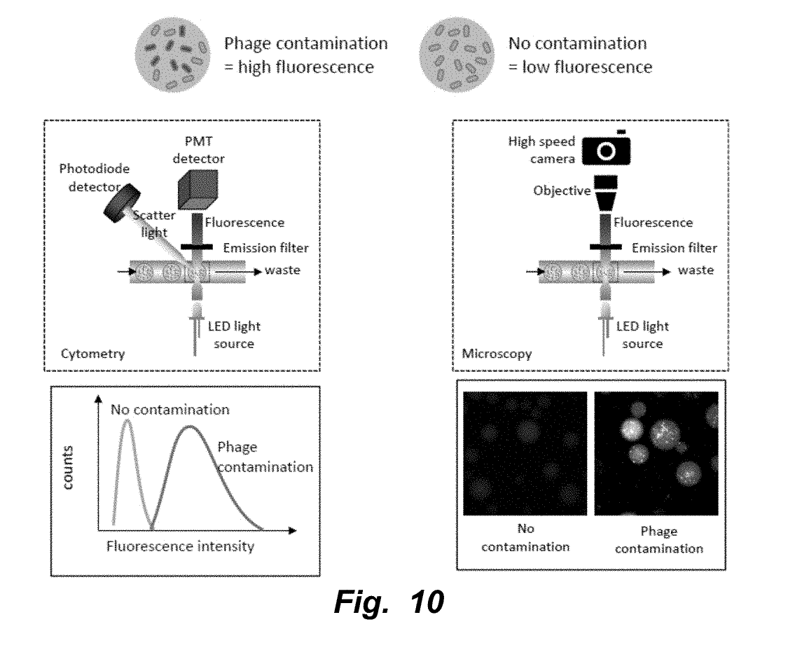

Droplets of the inner aqueous phase (W1) are suspended into an oil phase (0) comprising an oil and a hydrophobic emulsifier with an HLB value of 4 or less, to yield a water-in-oil (W1/O) emulsion. The water-in-oil (W1/O) emulsion or microdroplets can be formed using any method in the art. In varying embodiments, the droplets of the inner aqueous phase are delivered or suspended into the oil using a needle, e.g., having a needle gauge in the range of about 18 G to about 34 G, e.g., 18 G, 19 G, 20 G, 21 G, 22 G, 23 G, 24 G, 25 G, 26 G, 27 G, 28 G, 29 G, 30 G, 31 G, 32 G, 33 G or 34 G, depending on the desired size of suspended droplet. In varying embodiments, the needle can have a beveled or blunt end. As described herein, the needle-in-tube method for forming water-in-oil emulsions is known in the art. In varying embodiments, water-in-oil (W1/O) emulsion or microdroplets are formed in the inner space of a tubing, wherein a channel containing the fluidic flow of the inner aqueous phase (W1) suspension flows into the confluence or junction of two channels of fluidic flow of the oil phase such that when the inner aqueous phase (W1) meets the confluence of the fluidic flow of the two channels of the oil phase, microdroplets of a water-in-oil (W1/O) emulsion are formed. This is depicted in the portable device of FIG. 9. In varying embodiments, the droplets can have an average diameter in the range of about 50 μm to about 300 μm, e.g., in the range of about 50 μm to about 100 μm.

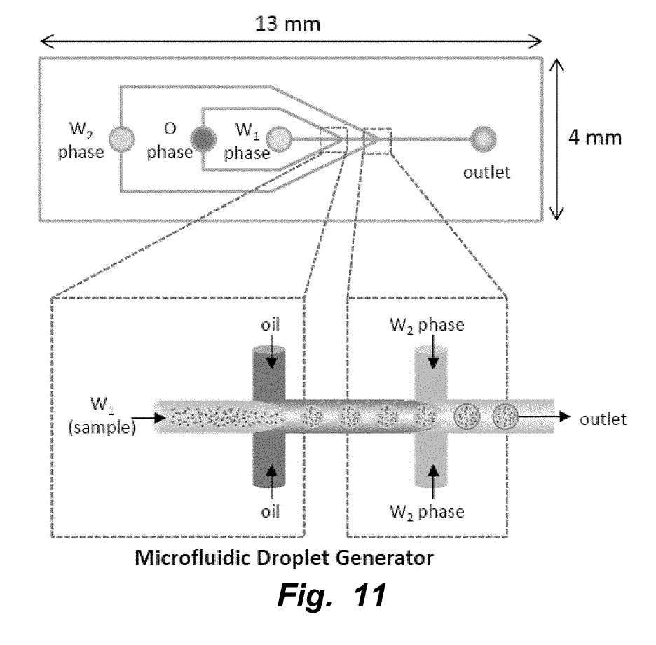

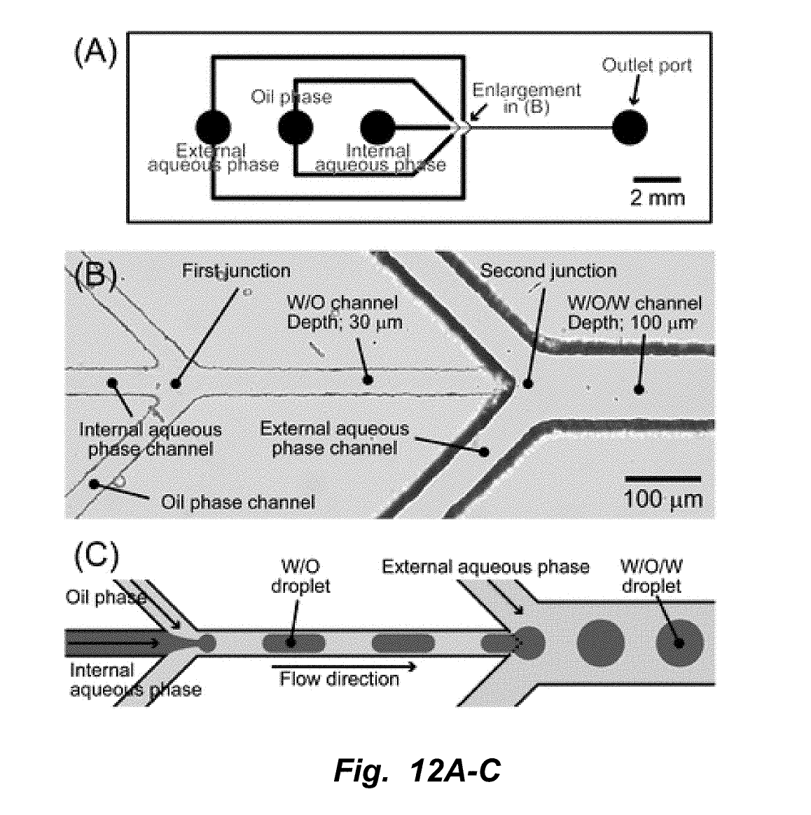

In varying embodiments, the inner aqueous phase (W1) microdroplets are delivered or suspended into the oil phase via a microfluidic lumen or channel in a microfluidic device, e.g., at a junction in the microfluidic device where the fluidic flow from a channel or lumen containing the inner aqueous phase (W1) flows into the fluidic flow of one or more channels or lumens containing the oil phase. See, e.g., the illustrative microfluidic lumen or channel configurations of a microfluidic device depicted in FIGS. 11-12, wherein the channel or lumen containing the inner aqueous phase (W1) flows into the junction of two channels or lumens (e.g., in an inverted Y or T configuration) containing the oil phase. Microdroplets form within the confluence of the fluidic flow of the three channels or lumens. The diameter of the microdroplets formed within the oil phase can be adjusted according to the inner diameter of the lumen containing the inner aqueous phase (W1).

Generally, the oil in the oil phase is liquid at 25-30° C. In varying embodiments, the oil in the oil phase is selected from mineral oil, canola oil, olive oil, corn oil, sunflower oil, safflower oil, peanut oil, coconut oil and fluorinated oils (e.g., perfluorodecalin).

With respect to the hydrophobic emulsifier, any hydrophobic emulsifier with a hydrophilic lipophilic balance (HLB) value of 4 or lower can be used. In varying embodiments, the emulsifier is a polyglycerol ester of fatty acid. In varying embodiments, the hydrophobic emulsifier comprises polyglycerol polyricinoleate (PGPR). Other emulsifiers that can find use in the oil phase include without limitation, e.g., Glycol Distearate (HLB=1); Sorbitan Trioleate (HLB=1.8); Propylene Glycol Isostearate (HLB=2.5); Glycol Stearate (HLB=2.9); Sorbitan Sesquioleate (HLB=3.7); Glyceryl Stearate (HLB=3.8); and Lecithin (HLB=4).

c. Forming Water-in-Oil-in-Water (W1/O/W2) Emulsion

The water-in-oil (W1/O) emulsion or microdroplets are then mixed in an outer aqueous phase (W2) comprising at least one water soluble emulsifier, yielding a water-in-oil-in-water (W1/O/W2) emulsion comprising bacterial cells. In varying embodiments, the outer aqueous phase can comprise water alone or an aqueous salt buffer, e.g., having a salt concentration in the range of about 10 μM to about 1.0 M.

With respect to the water soluble emulsifier in the outer aqueous phase (W2), in varying embodiments, the water soluble emulsifier in the outer aqueous phase (W2) comprises an HLB value greater than 7. The water soluble emulsifier in the outer aqueous phase (W2) may or may not be a mixture of emulsifying components. In varying embodiments, the water soluble emulsifier of the outer aqueous phase (W2) comprises a mixture of a bile salt, a zwitterionic detergent and a nonionic detergent. In varying embodiments, the outer aqueous phase (W2) comprises one or more bile salts, lecithin and Tween 20. In varying embodiments, the water soluble emulsifier of the outer aqueous phase (W2) comprises one or more emulsifiers selected from Lecithin; PEG-8 Dioleate (HLB=8); Sodium Stearoyl Lactylate (HLB=8.3±1); Sorbitan Laurate (HLB=8.6±1); PEG-40 Sorbitan Peroleate (HLB=9±1); Lecithin (HLB=9.7±1); Laureth-4 (HLB=9.7±1); PEG-7 Glyceryl Cocoate (HLB=10); PEG-20 Almond Glycerides (HLB=10); Linoleamide DEA (HLB=10); PEG-25 Hydrogenated Castor Oil (HLB=10.8); Stearamide MEA (HLB=11); Glyceryl Stearate and/or PEG-100 Stearate (HLB=11); Polysorbate 85 (HLB=11); PEG-7 Olivate (HLB=11); Cetearyl Glucoside (HLB=11); PEG-8 Oleate (HLB=11.6); Polyglyceryl-3 Methyglucose Distearate=12 Oleth-10 (HLB=12.4); Oleth-10/Polyoxyl 10 Oleyl Ether NF (HLB=12.4); Ceteth-10 (HLB=12.9); PEG-8 Laurate (HLB=13); Cocamide MEA (HLB=13.5); Polysorbate 60 NF (HLB=14.9); Polysorbate 60 (HLB=14.9); Polysorbate 80 (HLB=15); Isosteareth-20 (HLB=15); PEG-60 Almond Glycerides (HLB=15); Polysorbate 80 NF (HLB=15); PEG-20 Methyl Glucose Sesquistearate (HLB=15); Ceteareth-20 (HLB=15.2); Oleth-20 (HLB=15.3); Steareth-20 (HLB=15.3); Steareth-21 (HLB=15.5); Ceteth-20 (HLB=15.7); Isoceteth-20 (HLB=15.7); Polysorbate 20 (HLB=16.7); Polysorbate 20 NF (HLB=16.7); Laureth-23 (HLB=16.9); PEG-100 Stearate (HLB=18.8); Steareth-100 (HLB=18.8); PEG-80 Sorbitan Laurate (HLB=19.1).

The water-in-oil-in-water (W1/O/W2) emulsion or microdroplets can be formed using any method in the art. In varying embodiments, water-in-oil-in-water (W1/O/W2) emulsion or microdroplets are formed in the inner space of a tubing, wherein a channel containing the fluidic flow of the oil phase containing water-in-oil (W1/O) emulsion microdroplets flows into the confluence or junction of two channels of fluidic flow of the outer aqueous phase such that when the water-in-oil (W1/O) emulsion microdroplets meet the confluence of the fluidic flow of the two channels of the outer aqueous phase, microdroplets of a water-in-oil (W/O) and/or water-in-oil-in-water (W1/O/W2) emulsion are formed. This is depicted in the portable device of FIG. 9. In varying embodiments, the droplets can have an average diameter in the range of about 50 μm to about 300 μm, e.g., in the range of about 50 μm to about 100 μm.

In varying embodiments, the water-in-oil (W1/O) emulsion microdroplets are delivered or suspended into the outer aqueous phase via a microfluidic lumen or channel in a microfluidic device, e.g., at a junction in the microfluidic device where the fluidic flow from a channel or lumen containing the water-in-oil (W1/O) emulsion microdroplets in the oil phase flows into the fluidic flow of one or more channels or lumens containing the outer aqueous phase. See, e.g., the illustrative microfluidic lumen or channel configurations of a microfluidic device depicted in FIGS. 11-12, wherein the channel or lumen containing the water-in-oil (W1/O) emulsion microdroplets in the oil phase flows into the junction of two channels or lumens (e.g., in an inverted Y or T configuration) containing the outer aqueous phase to form water-in-oil-in-water (W1/O/W2) emulsion or microdroplets. Microdroplets form within the confluence of the fluidic flow of the three channels or lumens. The diameter of the water-in-oil-in-water (W1/O/W2) emulsion microdroplets formed within the outer aqueous phase can be adjusted according to the inner diameter of the lumen containing the oil phase and the outer aqueous phase. Generally, droplet size will depend on the fluidic channel diameters and flow rates at each channel.

d. Detecting Changes in the Cell Viability Dye

Changes in cell viability can be detected using various dye or reporter molecules. Signal from the cell viability dye or reporter encapsulated in the water-in-oil (W/O) water-in-oil-in-water (W1/O/W2) emulsion or microdroplets is detected as an indicator of the presence of bacteriophage in the bacterial cell population. The cell viability dye can be detected using any method known in the art appropriate to the kind of dye employed, e.g., a fluorophore or a colorimetric dye. In varying embodiments, the signal from the cell viability dye is detected by visual inspection. In varying embodiments, the signal from the cell viability dye is detected by optical microscopy. In varying embodiments, the signal from the cell viability dye is detected by flow cytometry. In varying embodiments, the signal from the cell viability dye is detected without employing flow cytometry, nucleic acid amplification or impedance spectroscopy.

In varying embodiments, the detecting step is performed in comparison to a control comprising the inner aqueous phase without bacteriophage.

3. Portable Bacteriophage Detection Device

Further provided is a portable device for detection of the presence of bacteriophage in a bacterial culture or mixture, e.g., using the methods described herein. In varying embodiments, the device comprises a tubing in fluid communication from the upstream to downstream direction, with (i) a fluidic droplet generator, (ii) an incubator and (iii) a detector. Upstream of the fluidic droplet generator, e.g., at the upstream end, the tubing is in fluid communication with a sample reservoir, which can be a syringe. The sample reservoir can contain the inner aqueous phase, with embodiments as described above and herein, e.g., a bacterial cell population suspended in a water soluble emulsifier having an HLB value of 10 or greater.

In varying embodiments, the tubing within the fluidic droplet generator comprises a first upstream syringe comprising a needle pierced into the inner space or inner lumen or inner channel of the tubing, wherein the inner space of the first syringe contains an oil phase comprising an emulsifier; and a second downstream syringe containing a needle pierced into the inner space or inner lumen or inner channel of the tubing downstream from the needle of the first syringe, wherein the inner space of the second syringe comprises an aqueous phase comprising at least one detergent. The first syringe can contain an oil phase, with embodiments as described above and herein, e.g, an oil and an emulsifier having an HLB value of 4 or lower. The second syringe can contain the outer aqueous phase, with embodiments as described above and herein, e.g., an aqueous solution comprising a water soluble emulsifier having an HLB value of about 7 or greater, e.g., that can be a mixture comprising a bile salt, a zwitterionic detergent and a nonionic detergent.

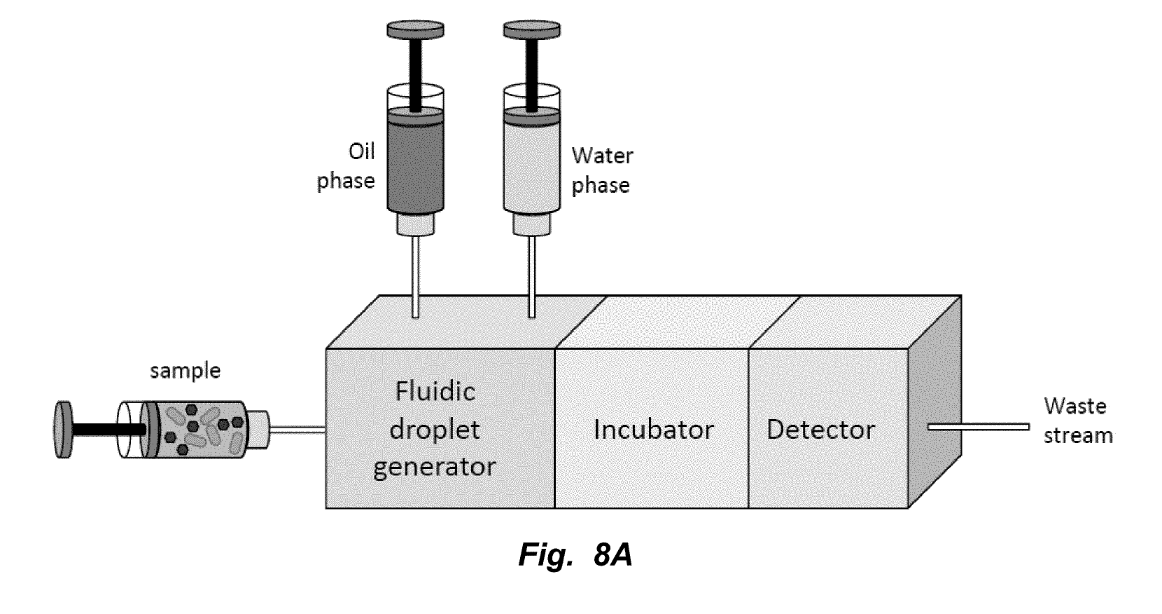

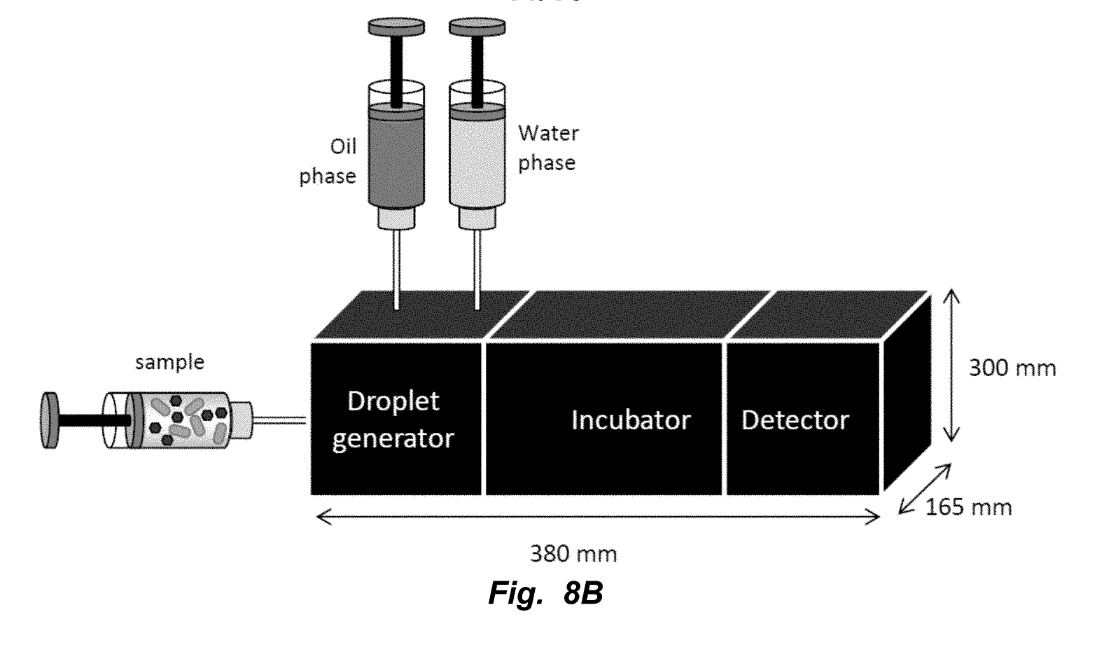

In varying embodiments, the incubator can hold a preselected or predetermined temperature in the range of about 4° C. to about 50° C., e.g., in the range of about 25° C. to about 37° C. In varying embodiments, the detector can detect a fluorescent signal and/or a colorimetric signal. In varying embodiments, one or more of the sample reservoir, the first syringe and the second syringe automatically deliver fluid. For example, the device can further comprise a controller in electrical and/or mechanical communication with one or more of the sample reservoir (which can be a syringe), the first syringe and the second syringe such that the plungers of the syringes can automatically depress to incrementally dispense the inner aqueous phase, the oil phase and/or the outer aqueous phase into the tubing. Fluid flows through the tubing in the upstream to downstream direction from the sample reservoir to the inlet of the first syringe; from the inlet of the first syringe to the inlet of the second syringe; and from the inlet of the second syringe to the incubator, the detector and then the outlet. In varying embodiments, the portable device weighs less than 10 kg, e.g., less than about 9 kg, 8 kg, 7 kg, 6 kg, 5 kg, 4 kg, 3 kg, 2 kg, 1 kg, or less. In varying embodiments, the device has a desk or table footprint of less than about 200 in², e.g., less than about 190 in², 180 in², 170 in², 160 in², 150 in², 140 in², 130 in², 120 in², 110 in², 100 in², or less. In varying embodiments, the inner space or inner lumen or inner channel of the tubing has a diameter in the range of about 1/32 (0.03125) inches to about 1/16 (0.0625) inches. In varying embodiments, the needle of the first syringe and/or the second syringe has a gauge from 18 G to 34 G, e.g., from 25 G to 30 G, e.g., e.g., 18 G, 19 G, 20 G, 21 G, 22 G, 23 G, 24 G, 25 G, 26 G, 27 G, 28 G, 29 G, 30 G, 31 G, 32 G, 33 G or 34 G. In varying embodiments, the device is as depicted in FIGS. 8, 9 and/or 10.

In varying embodiments, the methods described herein are performed using a portable bacteriophage detection device, as described above.

4. Microfluidic Bacteriophage Detection Device

Further provided is a microfluidic device for creating water-in-oil-in-water (W1/O/W2) emulsion droplets. In varying embodiments the microfluidic device comprises one or more units or modules of channels for creating water-in-oil (W/O) and/or water-in-oil-in-water (W1/O/W2) emulsion droplets, one unit comprising:
i) a first inlet in fluid communication with a first lumen or channel, the first inlet and first lumen or channel comprising an inner aqueous phase;
ii) a second inlet in fluid communication with a second lumen or channel, the second inlet and second lumen or channel comprising an oil phase, wherein the second lumen or channel is in fluid communication with the first lumen or channel;
iii) a third inlet in fluid communication with a third lumen or channel, the third inlet and third lumen or channel comprising an outer aqueous phase, wherein the third lumen or channel is in fluid communication with the first lumen or channel, wherein the third lumen or channel connects with the first lumen or channel downstream of where the second lumen or channel connects with the first lumen or channel; and
iv) an outlet for collecting water-in-oil-in-water (W1/O/W2) emulsion droplets, wherein the outlet is in fluid communication with the first lumen or channel. In varying embodiments, the outlet is in fluid communication with an incubator and a detector. The embodiments of the inner aqueous phase, oil phase and outer aqueous phase are as described above and herein. In varying embodiments, the second lumen or channel extends bidirectionally in first and second branches from the second inlet and forms a junction on opposing sides of the first lumen or channel, such that the fluid flows into the first lumen or channel from first and second branches of the second channel. Similarly, in varying embodiments, the third lumen or channel extends bidirectionally in first and second branches from the third inlet and forms a junction on opposing sides of the first lumen or channel, such that the fluid flows into the first lumen or channel from first and second branches of the third channel. In varying embodiments, the junction of the first lumen or channel and the first and second branches of the second lumen or channel is in the shape of a Y or T. Independently, in varying embodiments, the junction of the first lumen or channel and the first and second branches of the third lumen or channel is in the shape of a Y or T. In varying embodiments, the inner diameters of the first, second and third lumens are from about 30 μm to about 150 μm, e.g., about 30 μm, 40 μm, 50 μm, 60 μm, 70 μm, 80 μm, 90 μm, 100 μm, 110 μm, 120 μm, 130 μm, 140 μm, 150 μm. In varying embodiments, the configuration of the microfluidic device is as depicted in FIGS. 11-12. Generally, any co-axial and cross-flow (e.g., T- and/or Y-junction) setup can be used. In varying embodiments, the microfluidic device is a chip that is about the size of a microscope slide. In varying embodiments, the microfluidic unit for creating water-in-oil (W/O) and/or water-in-oil-in-water (W/O/W) emulsions has an area of about 100 mm² or less, e.g., an area of about 95 mm², 90 mm², 85 mm², 80 mm², 75 mm², 70 mm², 65 mm², 60 mm², 55 mm², 50 mm², or less.

In varying embodiments, the methods described herein are performed using a microfluidic bacteriophage detection device, as described above.

5. Kits

Further provided are kits. In varying embodiments, the kits can comprise vials containing an inner aqueous phase, an oil phase and an outer aqueous phase, as described above. In varying embodiments, the kits can comprise vials containing an inner aqueous phase and an oil phase, as described above. In varying embodiments, the kits can further comprise a portable device for the preparation of water-in-oil-in-water emulsions, as described above and herein. In varying embodiments, the kits can further comprise a microfluidic device for the preparation of water-in-oil-in-water emulsions, as described above and herein.

### EXAMPLES

The following examples are offered to illustrate, but not to limit the claimed invention.

### Example 1

### Rapid Detection of Bacteriophages in Starter Culture Using Water-in-Oil-in-Water Emulsion Microdroplets

### MATERIALS AND METHODS

Materials.

Clear Tygon PVC tubing ( 1/16″ ID and 3/16″ OD) was from McMaster-Carr. Kendall MonoJect hypodermic needles (27 G×1.5), 5 mL and 1 mL disposable syringes, phosphate buffered saline (PBS) were from Thermo Fisher Scientific (Waltham, Mass.). Mineral oil, bile salt, Tween 20 and propidium iodide (PI) were from Sigma Aldrich (St. Louis, Mo.). Polyglycerol polyricinoleate (PGPR 4175) was from Palsgaard (Morris Plains, N.J.). Whey protein isolate (WPI) was a generous gift from Professor John M. Krochta from the University of California, Davis. Low melting lecithin (ALCOLEC® PC75) was also a gift from American Lecithin Inc. (Oxford, Conn.). The SYBR Green I and BODIPY 665 dyes were from Life Technologies (Carlsbad, Calif.). All chemicals were used as is without further purification.

Bacterial Culture and Growth Conditions.

E. coli BL21 (ATCC, #BAA-1025, Manassas, Va.) was used as a model bacteria in this study and grown according to the manufacturer's protocol. Briefly, the stock bacteria was streaked on LB agar plate and grown overnight at 37° C. to obtain isolated bacterial colonies. Fresh bacterial cultures were prepared weekly by picking one bacterial colony from the LB agar plate and grown overnight in 50 mL LB media at 37° C. and shaken at 250 rpm. Then, an aliquot of this overnight culture was grown in 5 mL LB media until late log-phase (OD600˜0.8, 10⁸ CFU/mL) and used for encapsulation.

Phage Propagation.

The T7 phages (ATCC, #BAA-1025-B2, Manassas Va.) were propagated on LB agar plates. Briefly, an aliquot of stock T7 phages (˜10⁴ PFU/mL final concentration) were mixed with 300 μL of E. coli BL21 added to 3.5 mL molten agar (0.3% w/v). The solution was inverted once to mix and poured over a LB agar plate and incubated overnight at room temperature to form plaques. The soft agar layer was gently scraped off, resuspended with 10 mL of 1×TBS-Mg (50 mM Tris, 150 mM NaCl and 10 mM MgCl₂), and placed on a shaking incubator for 30 min at 250 rpm to allow the phages to diffuse out of the agar. The agar/phage mixture was then centrifuged at 10,000 rpm for 10 min to remove bacterial and agar debris and filter using a 0.22 μm syringe filter. The phages were stored at 4° C. until use.

Compositions of the (W₁/O/W₂) Emulsion Microdroplets.

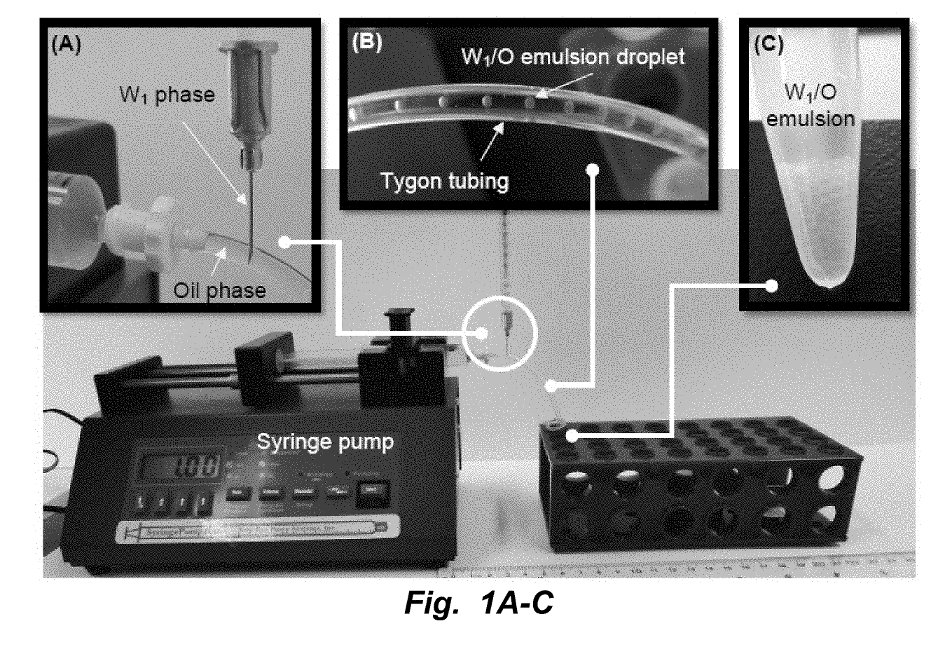

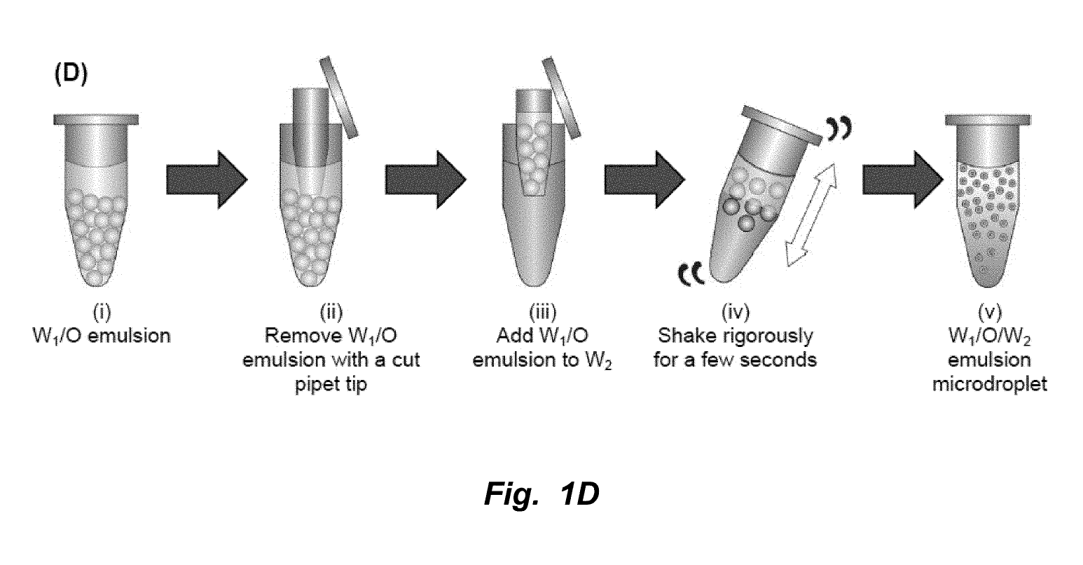

The inner aqueous phase (W1) was composed of: 5% (w/w) WPI, E. coli BL21 bacteria (10⁸ CFU/mL), and PI dye (1 μg/mL final concentration). Different titers of T7 phages (10², 10⁴ and 10⁶ PFU/mL) were added to the W1 phase no more than 5 min prior to W1/O generation. The oil phase (0) was composed of 6% (w/w) PGPR and 94% mineral oil. A lipophilic BODIPY 665 dye (0.025 μg/mL) was added to visualize the O phase. The outer aqueous phase (W2) was made by first mixing 1.5% (w/w) lecithin with 0.5% (w/w) bile salts in water, and the solution was stirred rigorously at 40° C. for 1 hr to dissolve the lecithin. Next, the solution was probe sonicated using a Qsonica sonicator (Model Q55, 50 W power, 20 kHz frequency; Newton, Conn.) to generate smaller lecithin/bile vesicles. The probe sonicator was set at 50% power, and manually pulse 5 times on a 3 sec on/3 sec off cycle. Then, Tween 20 (0.4 wt. % final concentration) was added to the sonicated lecithin/bile salt solution to make up the final W2 phase (FIG. 1).

Generation of the W₁/O Emulsion Using a Needle-in-Tube Setup.

The first W1/O emulsion was synthesized by injecting W1 solution into a continuous flow of O phase (FIG. 1). The O phase was delivered using a syringe pump (Model NE-300, New Era Pump System, Inc.; Farmingdale, N.Y.) and the flow rate was set to 6 mL/min (FIG. 1). The W1 phase was manually injected into the O phase using a 1 mL disposable syringe fitted with a 27 G hypodermic needle (Inset A in FIG. 1). The W1 solution filled syringe was tapped to remove air bubbles and the plunger was pushed forward to relieve these air bubbles. The 27 G needle on the W1 syringe was then pierced into the Tygon tubing at a distance of approximately 1 cm from the 5 mL syringe tip (Inset A in FIG. 1). The depth of the needle pierced into the Tygon tubing did not affect the final W1/O emulsion generation. The syringe pump (carrying the O phase syringe) was started once the first drop of W1 solution was manually injected into the Tygon tubing through the needle. A constant injection rate was maintained to continuously generate stable W1/O emulsion droplet and the process was stopped after about 20 sec (Inset B in FIG. 1). At this point, approximately 1 mL of W1/O emulsion was collected in the 1.5 mL microcentrifuge tube (Inset C in FIG. 1).

Preparation of the W₁/O/W₂ Emulsion Microdroplets.

A 1 mL solution of W2 was prepared in a second 1.5 mL microcentrifuge tube. Once the W1/O emulsion had settled to the bottom of the tube, 400 μL of the W1/O emulsion was pipetted into the W2 solution using a cut pipet tip (FIG. 1). Then, the microcentrifuged tube was shaken rigorously for a few seconds to generate the W1/O/W2 emulsion microdroplets (FIG. 1).

Visualization of the W1/O/W2 Emulsion Microdroplets Using Optical Microscopy.

An aliquot of the SYBR Green I dye (5 μg/mL) was added to the W1 to stain the bacteria and BODIPY 665 was added to the O phase to visualize the W1 and O phase, respectively. The fluorescence excitation and emission of the SYBR Green dye in the W1 phase were 480/30 nm and 535/40 nm, respectively (Olympus). The fluorescence excitation and emission of the BODIPY 665 dye in the O phase were 640/20 nm and 680/30 nm, respectively (Olympus). The brightfield and fluorescence microscopy images of the W1/O/W2 emulsion microdroplets were taken using an Olympus IX-71 inverted fluorescence microscope with either a 4× or a 10× objective (Olympus UPlanFLN).

Detection of PI-Labeled Bacteria in the W1/O/W2 Emulsion Microdroplets.

To detect phage contamination, different concentrations of phages (10²-10⁶ PFU/mL) were incubated with 10⁸ CFU/mL of bacteria and 5 μg/mL of the PI dye in the W1 phase. The W1/O/W2 emulsion microdroplets were made using the methods described above, and the encapsulated bacteria and phages were incubated at 37° C. for 1 hr. For comparison, a non-encapsulated sample of bacteria (10⁸ CFU/mL) and phages (10⁶ PFU/mL) was also prepared as an aqueous suspension. Then, 40 μL of the W1/O/W2 emulsion microdroplets was added to 100 μL PBS in a 96-well plate. The fluorescence images of the W1/O/W2 emulsion microdroplets were taken using an Olympus IX-71 inverted fluorescence microscope with a 4× objective (Olympus UPlanFLN, NA=0.13). The PI dye-labeled bacteria in the W1 phase was excited using a 540/25 nm filter and the fluorescence emission was measured using a 605/55 nm filter (Olympus).

Image Processing and Analysis.

The fluorescence intensity of the PI dye was analyzed using ImageJ (http://rsbweb.nih.gov/ij/). Each image was filtered using a rolling ball filter (ball radius of 500 pixels) to eliminate background noise and correct for image tilt. All image were thresholded and set to 0-255 (min-max) pixel intensity. A region of interest (ROI) was drawn around each W1/O/W2 emulsion microdroplets from each fluorescence image, and the mean pixel intensity (MPI) of the ROI was determined using Image J.

Enumeration Ofphage Amplification.

The W1/O/W2 emulsion microdroplets were centrifuged at 13,200 rpm for 10 min to disrupt the emulsion. A clear phase separation between the oil and aqueous phase was obtained after centrifugation and the released phages were located in the aqueous phase. Titers of the released phages were enumerated using the standard plate plaque assay. Briefly, released phages were serially diluted with sterile PBS and added to 3 mL molten soft agar (3% w/v) with 300 μL of an overnight E. coli BL21 culture. The agar solution was poured over pre-warmed LB agar plate and incubated at 37° C. for 2 hr. Visible phage plaques on the LB agar plates were subsequently counted.

Statistical Analysis.

Three independent experiments were conducted and the mean pixel intensities (MPI) from at least 170 W1/O/W2 emulsion microdroplets were quantified for each sample. All data was reported as mean±SEM. A p-value of <0.05 using a Student's t-test was considered as statistically significant. The size distribution, boxplot of MPI and statistical analyses were determined using the Minitab 16 statistical software (Minitab, Inc., State College, Pa.).

### RESULTS

Encapsulation of Bacteria and Phages in W1/O/W2 Emulsion Microdroplets.

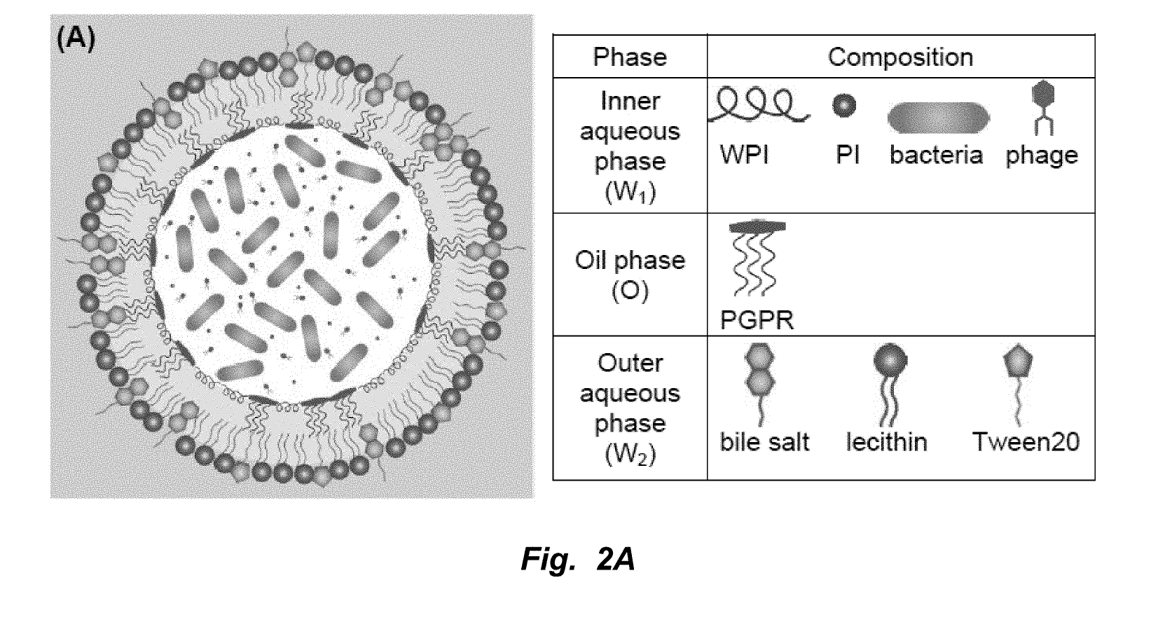

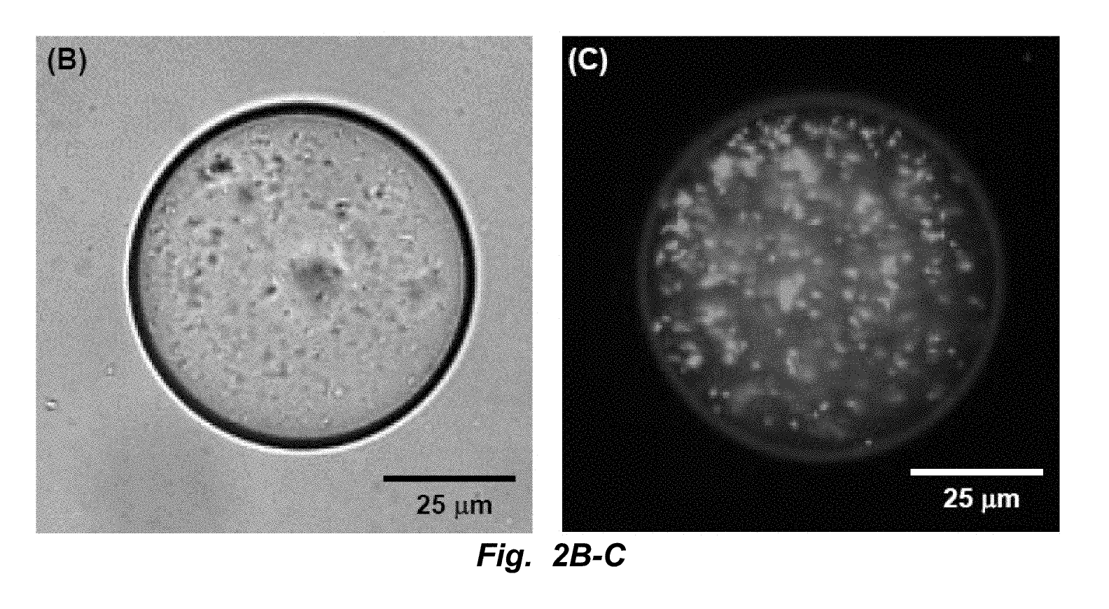

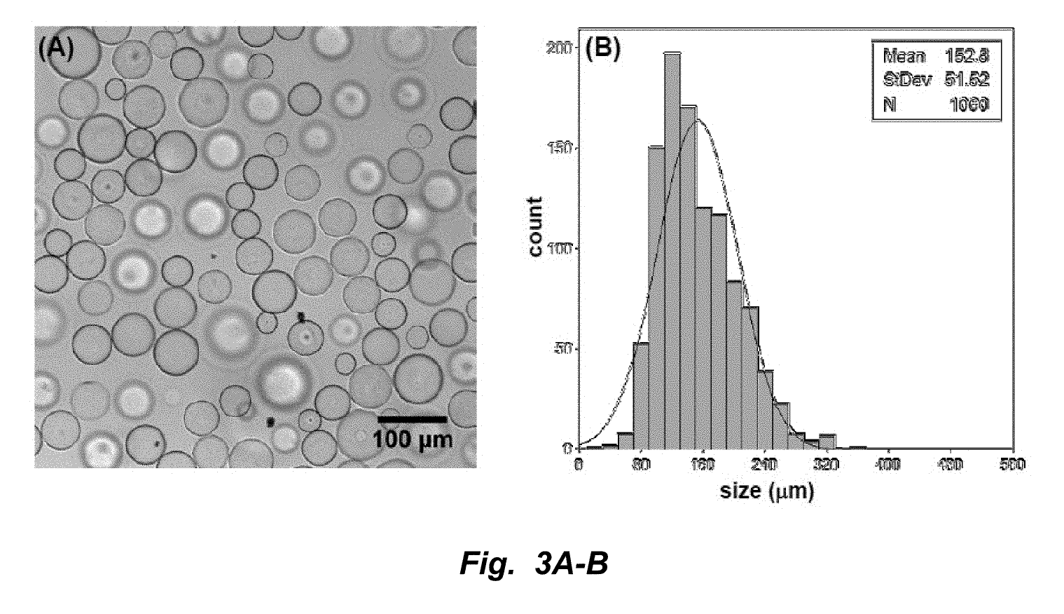

Bacteria and phages were encapsulated in W1/O/W2 emulsion microdroplets using a tubing-based setup (i.e., needle-in-tube) as demonstrated (FIG. 1). To visualize the W1/O/W2 emulsion microdroplets and to confirm the encapsulation of bacteria, an optical imaging approach was used. The brightfield and fluorescence images showed that the bacteria, labeled with SYBR Green (green) were encapsulated within the W1 phase, and the PGPR stabilized O phase, labeled with BODIPY 665 (blue) was seen surrounding each W1/O/W2 emulsion microdroplets (FIGS. 2B and C). The average size of the W1/O/W2 emulsion microdroplets was 152±50 μm from a total number of 1,060 microdroplets analyzed (FIGS. 3A and B). It is noteworthy that due to constant movement of the emulsion microdroplets, some emulsion microdroplets were outside the focus plane of the objective and appeared to have blurred boundaries compared to the ones that were in focus.

W1/O/W2 Emulsion Microdroplets Improve Signal Contrast for Detection.

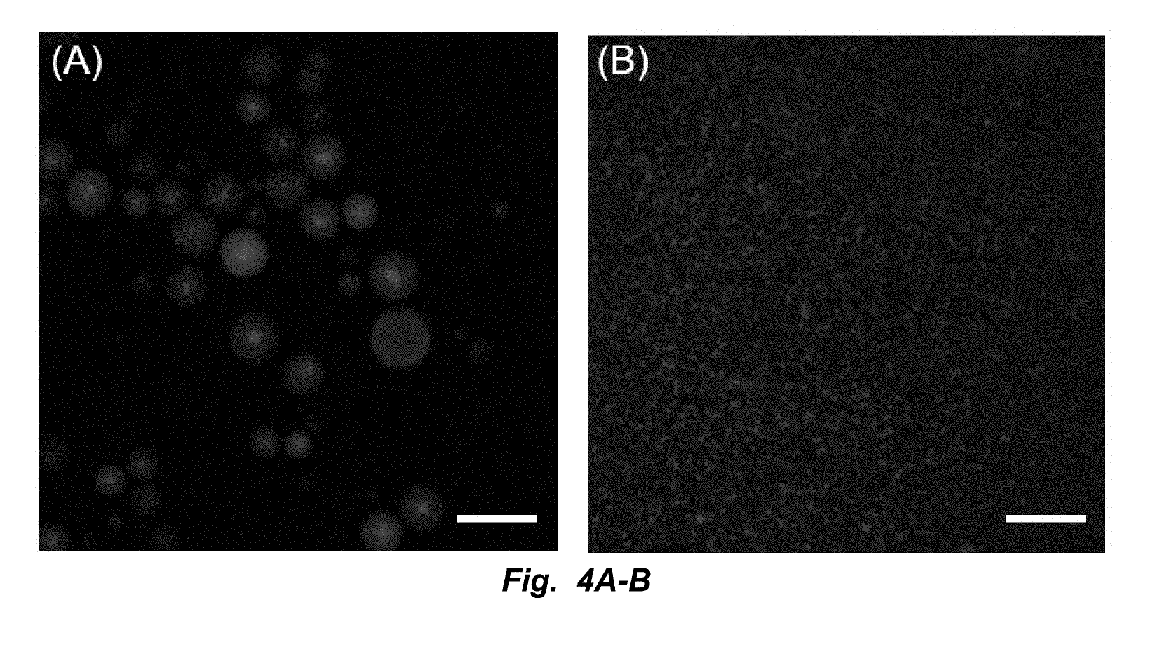

To demonstrate the effect of signal enhancement using the W1/O/W2 emulsion microdroplets, a direct comparison between the encapsulated (W1/O/W2) and non-encapsulated (aqueous suspension) sample was performed (FIG. 4). After 1 hr of incubation, a substantial amount of phage-infected bacteria could be detected in the encapsulated sample, as the fluorescence signal inside each W1/O/W2 emulsion microdroplet was considerably higher compared to the background noise (FIG. 4A). However, the increase in fluorescence signal in the non-encapsulated sample was not as easily detected due to lack of distinction between the background and signal fluorescence (FIG. 4B). Therefore, a good signal to background contrast of the phage-infected bacteria could be easily detected using the W1/O/W2 emulsion microdroplets due to the localized concentration of bacteria inside each microdroplet (FIG. 4A), compared to the same concentration of that were not encapsulated (FIG. 4B).

Qualitative Analysis of PI-Labeled Bacteria in the W1/O/W2 Emulsion Microdroplets.

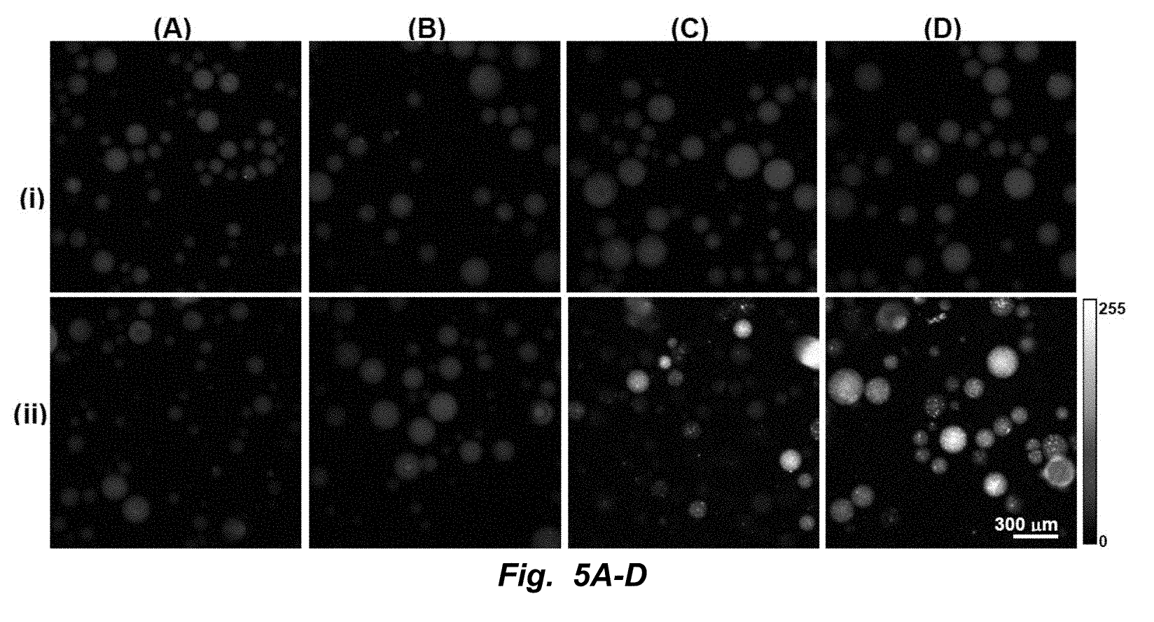

Since the T7 phages have diameters (60-65 nm) that are below the resolution for optical microscopy (34), an indirect approach was used to determine the levels of phages in the W1/O/W2 emulsion microdroplets (FIG. 5). In this approach, PI dye was used to label bacteria that have compromised cell membrane, thus a PI-labeled bacterium would be indicative of a phage infection (FIG. 5). Therefore, it is expected that the number of PI-labeled bacteria in the W1/O/W2 emulsion microdroplets would be proportional to the levels of phages in the microdroplets (FIG. 4). Phage titers of 10², 10⁴ and 10⁶ PFU/mL were incubated with 10⁸ CFU/mL BL21 bacteria for 1 hr at 37° C. At 0 hr, there were no visual differences in the fluorescence intensities across all W1/O/W2 emulsion microdroplet samples (FIG. 5Ai-Di). After an hour of incubation at 37° C., only a slight increase in fluorescence signal inside the W1/O/W2 emulsion microdroplets could be observed in the 10² PFU/mL phage sample (FIG. 5Bii). This slight increase in signal was visually comparable to the control sample, where no phages were added (FIG. 5Aii). On the other hand, the fluorescence signal inside the W1/O/W2 emulsion microdroplets of the 10⁴ (FIG. 5Cii) and 10⁶ PFU/mL (FIG. 5Dii) samples were visually higher compared to the control (FIG. 5Aii). In addition, a number of highly fluorescent granules were seen inside the W1/O/W2 emulsion microdroplets of these samples (FIG. 5Cii & Dii). These granulated structures were likely from the bacteria with significant membrane damage, as more PI dye could permeate the cell membrane and bind to the DNA. Overall, the qualitative analysis suggested that the number of PI-labeled bacteria were visually higher in the 10⁴ and 10⁶ PFU/mL phage sample compared to the 10² PFU/mL sample and the control.

Quantification of MPI Inside W1/O/W2 Emulsion Microdroplets.

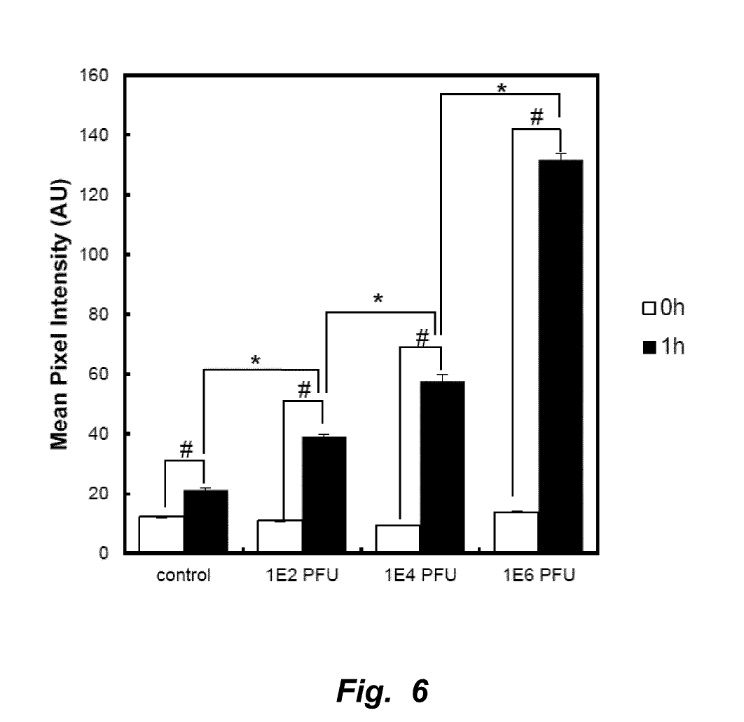

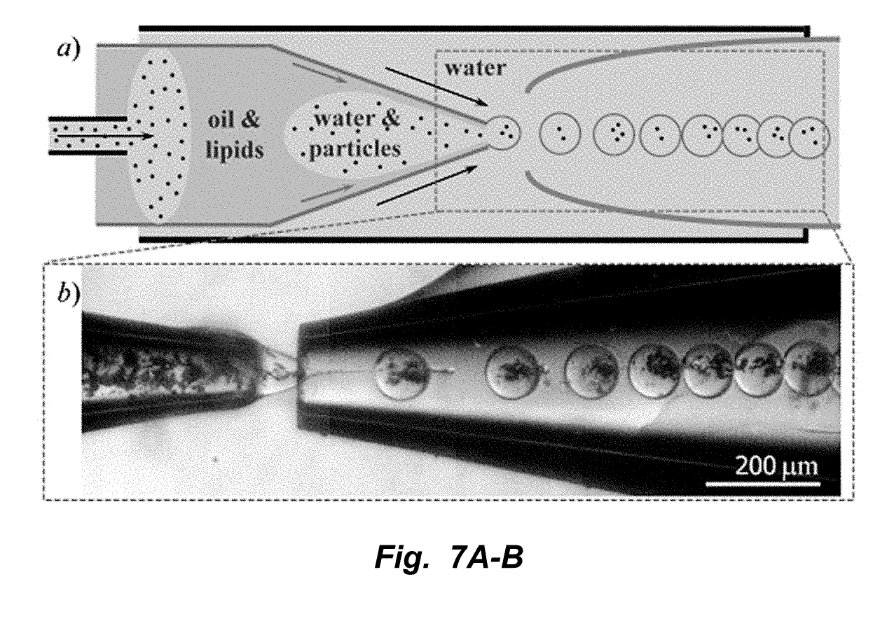

The increase in MPI inside the W1/O/W2 emulsion microdroplet for each sample was further quantified and compared (FIG. 6). At time 0, the MPI was 12±0.3, 11±0.2, 9±0.1, and 14±0.2, for the control, 10², 10⁴ and 10⁶ PFU/mL phage samples, respectively (FIG. 6, white bars). No significant differences were measured between any samples at t=0 hr. After 1 hr of incubation, a significant increase in MPI was determined within each sample (#p<0.001) and between all samples (*p<0.001) (FIG. 6, black bars). The MPI for the control increased to 21±0.6, while the MPI for the 10², 10⁴ and 10⁶ PFU/mL samples increased to 39±1.0, 56±2.2, and 132±2.2, respectively (FIG. 6, black bars). The increase in MPI in the control could be attributed the intercalation of the PI dye with other nucleic acids present in the LB media, secreted or released from the bacteria. Despite the small but significant increase in background fluorescence, results from the quantitative analyses demonstrated that 10² PFU/mL phages could be detected using this W1/O/W2 emulsion microdroplet approach with optical microscopy.

Enumeration of Amplified Phages Inside the W1/O/W2 Emulsion Microdroplets.

The number of amplified phages in the W1/O/W2 emulsion microdroplets was enumerated using a standard plate plaque assay (Table 1). A 3 log increase in phage count was determined for the 10⁴ and 10⁶ PFU/mL initial phage concentration samples (Table 1). On the other hand, incubation with 10² PFU/mL of initial phage concentration resulted in a 4 log increase in phage count (Table 1). The level of phage amplification at each starting phage titer was comparable to a standard phage propagation system, where the bacteria and phages were incubated together in a bulk solution (Table 1).

TABLE 1
Enumeration of amplified phages in the W₁/O/W₂
emulsion microdroplets and in solution.

| | Amplified phages | Amplified phages |
|---|---|---|
| Starting phage | in W/O/W | in solution |
| (log₁₀ PFU/mL) | (log₁₀ PFU/mL ± SD) | (log₁₀ PFU/mL ± SD) |
| 6 | 9.2 ± 0.4 | 8.6 ± 0.1 |
| 4 | 7.6 ± 0.2 | 7.9 ± 0.4 |
| 2 | 6.5 ± 0.7 | 6.0 ± 0.2 |

Mean ± SD, N = 3.

### DISCUSSION

In this study, a rapid method to detect the presence of phages in a model bacterial culture was described. The encapsulation of bacteria and phages in W1/O/W2 emulsion microdroplets used in this study were produced by a simple and inexpensive setup using a syringe needle and a piece of tubing (FIG. 1). While the smaller and monodispersed emulsion microdroplets generated using microfluidics are very attractive for PCR and single cell analyses (12-17, 35), the commercialization of this technology has been limited due to scale up challenges including sampling of multiple culture vats and larger sample volumes most food and pharmaceutical industries need (26). In the fermentation industry, routine screening for phage contamination in the starter culture and raw material is needed to prevent spoilage of large vats of products. Therefore, from an economic standpoint, the needle-in-tube method offers a simpler and cheaper alternative to the chip-based microfluidics.

The microdroplets generated using the simple needle-in-tube setup can produce O/W emulsion (31) and polymeric microbeads (30, 33) with sizes ranging from 200-600 μm. Consistent with previous studies, the W1/O/W2 emulsion microdroplets generated in this study using the needle-in-tube method had an average diameter of 150 μm (FIG. 3). Moreover, previous studies have suggested that the uniformity and final size of the microdroplet could be further improved by adjusting the flow rates of the continuous phase (29, 32, 36, 37), and the viscosities of the water and oil phases (36, 37).

Given the high encapsulation efficiency6, high local concentration (38) and success of phage amplification in microdroplets demonstrated in this study and in prior studies (16, 17), it is envisioned that the W/O/W emulsion microdroplets could be used over a large concentration range of the encapsulant. This encapsulation system would also be especially useful when the concentration of the target analyte (e.g., phage) is low. To detect the presence of phages in a bacterial starter culture, three different titers of phages (10², 10⁴ and 10⁶ PFU/mL) were encapsulated with 10⁸ CFU/mL of bacteria in a W1/O/W2 emulsion microdroplet (FIG. 4). The levels of phages chosen were based on the current range of detection limit using traditional plaque assay (39, 40) (20-25 PFU/mL) and molecular based assays including PCR (1, 2, 4, 10) (10⁴-10⁷ PFU/mL) and flow cytometry (11) (10⁵ PFU/mL). Since the diameter of an individual phage of 60-65 nm (34) is below the resolution for optical microscopy, an indirect approach was employed in this study to detect phage contamination. In this approach, the phage contamination was detected by the positive labeling of bacteria with a membrane impermeable dye, PI (FIG. 4). Due to the high encapsulation efficiency of this technique, the localized fluorescence intensity inside the W1/O/W2 emulsion microdroplets provided a good signal to background contrast (FIG. 4). This, in turn enabled for a rapid qualitative and quantitative analysis of phage contamination using optical microscopy (FIG. 5).

Using this W1/O/W2 emulsion microdroplet and imaging approach, the phage detection could be achieved within one hour after phage infection (FIGS. 4 and 5). This detection method was 10-20 times faster compared to the traditional phage detection methods used in the fermentation industry such as plaque assay and activity test (2, 5, 8, 9). It was shown here that a phage-infection could be detected in one hour using T7 phages that have an amplification cycle of 25-40 min (41), it is expected that the detection time would vary depending on phage amplification cycle for different strains of bacteria/phage. Nevertheless, the phage amplification cycle could easily be enhanced by adding glycine (42) or antibiotics (43) to the culture media to encourage phage growth.

In current fermentation practice, a starter culture would be rotated when 10⁵ to 10⁶ PFU/mL of phages was detected in the whey (8). Using this approach, a detection limit of 10² PFU/mL phages could be achieved quantitatively (FIG. 5), which would allow for an earlier detection of phage contamination than current methods. This detection limit was at least two orders of magnitude better than the sensitivity of flow cytometry (11) and PCR (12-15)-based approaches. Moreover, unlike the PCR-based detection methods (1, 2, 4, 10), this microdroplet optical imaging method does not require an expensive PCR thermocylcer, enrichment of bacterial cells or phage DNA amplification. Furthermore, this technique also offers a qualitative sensitivity of 10⁴ PFU/mL phages, and could be easily accomplished by visual comparison of the images for the control and phage samples (FIG. 5). Thus, using a model bacteria and phage, it was demonstrated here that phage contamination could be detected in a bacterial culture. The methods find use for detection of bacteriophage contamination in bacterial strains relevant to the fermentation industry.

### CONCLUSION

In summary, this method is an attractive approach for the fermentation industry to rapidly detect phage contamination. It is a relatively simple and inexpensive method, and can be used with any strain of bacteria and phage, or in a mixed culture without a priori knowledge of the phage DNA sequence. Together, the results of this study demonstrated that phage infection in a bacterial culture can be detected using a simple W1/O/W2 emulsion microdroplet and imaging approach. Given the simplicity, high sensitivity and relatively low cost of this imaging approach compared to flow cytometry and PCR methods, it is useful for rapid detection or routine screening of phage contamination in starter culture in the fermentation industry.

### REFERENCES

1. del Rio, B.; Binetti, A.; Martin, M. C.; Fernindez, M.; Magadan, A.; Alvarez, M., Multiplex PCR for the detection and identification of dairy bacteriophages in milk. Food microbiology 2007, 24, (1), 75-81.
2. Marcó, M. B.; Moineau, S.; Quiberoni, A., Bacteriophages and dairy fermentations. Bacteriophage 2012, 2, (3), 149-158.
3. Jones, D. T.; Shirley, M.; Wu, X.; Keis, S., Bacteriophage Infections in the Industrial Acetone Butanol(AB) Fermentation Process. Journal of molecular microbiology and biotechnology 2000, 2, (1), 21-26.
4. Garneau, J. E.; Moineau, S., Bacteriophages of lactic acid bacteria and their impact on milk fermentations. Microbial cell factories 2011, 10, (Suppl 1), S20.
5. Campagna, C.; Villion, M.; Labrie, S. J.; Duchaine, C.; Moineau, S., Inactivation of dairy bacteriophages by commercial sanitizers and disinfectants. International journal of food microbiology 2014, 171, 41-47.
6. Coakley, M.; Fitzgerald, G.; Ros, R., Application and evaluation of the phage resistance- and bacteriocin-encoding plasmid pMRC01 for the improvement of dairy starter cultures. Applied and environmental microbiology 1997, 63, (4), 1434-1440.
7. del Rio, B.; Martin, M. C.; Ladero, V.; Martinez, N.; Linares, D. M.; Fernindez, M.; Alvarez, M. A., Bacteriophages in Dairy Industry: PCR Methods as Valuable Tools.
8. Durmaz, E.; Klaenhammer, T. R., A starter culture rotation strategy incorporating paired restriction/modification and abortive infection bacteriophage defenses in a single Lactococcus lactis strain. Applied and environmental microbiology 1995, 61, (4), 1266-1273.
9. Kleppen, H. P.; Bang, T.; Nes, I. F.; Holo, H., Bacteriophages in milk fermentations: diversity fluctuations of normal and failed fermentations. International Dairy Journal 2011, 21, (9), 592-600.
10. Binetti, A. G.; Capra, M. L.; Alvarez, M. A.; Reinheimer, J. A., PCR method for detection and identification of Lactobacillus casei/paracasei bacteriophages in dairy products. International journal of food microbiology 2008, 124, (2), 147-153.
11. Michelsen, O.; Cuesta-Dominguez, Á.; Albrechtsen, B.; Jensen, P. R., Detection of bacteriophage-infected cells of Lactococcus lactis by using flow cytometry. Applied and environmental microbiology 2007, 73, (23), 7575-7581.
12. Urabe, H.; Ichihashi, N.; Matsuura, T.; Hosoda, K.; Kazuta, Y.; Kita, H.; Yomo, T., Compartmentalization in a water-in-oil emulsion repressed the spontaneous amplification of RNA by Q3 replicase. Biochemistry 2010, 49, (9), 1809-1813.
13. Leng, X.; Zhang, W.; Wang, C.; Cui, L.; Yang, C. J., Agarose droplet microfluidics for highly parallel and efficient single molecule emulsion PCR. Lab on a Chip 2010, 10, (21), 2841-2843.
14. Tewhey, R.; Warner, J. B.; Nakano, M.; Libby, B.; Medkova, M.; David, P. H.; Kotsopoulos, S. K.; Samuels, M. L.; Hutchison, J. B.; Larson, J. W., Microdroplet-based PCR enrichment for large-scale targeted sequencing. Nature biotechnology 2009, 27, (11), 1025-1031.
15. Hindson, B. J.; Ness, K. D.; Masquelier, D. A.; Belgrader, P.; Heredia, N. J.; Makarewicz, A. J.; Bright, I. J.; Lucero, M. Y.; Hiddessen, A. L.; Legler, T. C., High-throughput droplet digital PCR system for absolute quantitation of DNA copy number. Analytical chemistry 2011, 83, (22), 8604-8610.
16. Matochko, W. L.; Ng, S.; Jafari, M. R.; Romaniuk, J.; Tang, S. K.; Derda, R., Uniform amplification of phage display libraries in monodisperse emulsions. Methods 2012, 58, (1), 18-27.
17. Derda, R.; Tang, S. K.; Whitesides, G. M., Uniform amplification of phage with different growth characteristics in individual compartments consisting of monodisperse droplets. Angewandte Chemie International Edition 2010, 49, (31), 5301-5304.
18. Juul, S.; Ho, Y.-P.; Koch, J.; Andersen, F. F.; Stougaard, M.; Leong, K. W.; Knudsen, B. R., Detection of single enzymatic events in rare or single cells using microfluidics. ACS nano 2011, 5, (10), 8305-8310.
19. Huebner, A.; Olguin, L. F.; Bratton, D.; Whyte, G.; Huck, W. T.; de Mello, A. J.; Edel, J. B.; Abell, C.; Hollfelder, F., Development of quantitative cell-based enzyme assays in microdroplets. Analytical chemistry 2008, 80, (10), 3890-3896.
20. Kelly, B. T.; Baret, J.-C.; Taly, V.; Griffiths, A. D., Miniaturizing chemistry and biology in microdroplets. Chemical Communications 2007, (18), 1773-1788.
21. Teh, S.-Y.; Lin, R.; Hung, L.-H.; Lee, A. P., Droplet microfluidics. Lab on a Chip 2008, 8, (2), 198-220.
22. Shah, R. K.; Shum, H. C.; Rowat, A. C.; Lee, D.; Agresti, J. J.; Utada, A. S.; Chu, L.-Y.; Kim, J.-W.; Fernandez-Nieves, A.; Martinez, C. J., Designer emulsions using microfluidics. Materials Today 2008, 11, (4), 18-27.
23. Nisisako, T.; Okushima, S.; Torii, T., Controlled formulation of monodisperse double emulsions in a multiple-phase microfluidic system. Soft Matter 2005, 1, (1), 23-27.
24. Huebner, A.; Sharma, S.; Srisa-Art, M.; Hollfelder, F.; Edel, J. B., Microdroplets: a sea of applications? Lab on a Chip 2008, 8, (8), 1244-1254.
25. Song, H.; Chen, D. L.; Ismagilov, R. F., Reactions in droplets in microfluidic channels. Angewandte chemie international edition 2006, 45, (44), 7336-7356.
26. Holtze, C., Large-scale droplet production in microfluidic devices—an industrial perspective. Journal of Physics D: Applied Physics 2013, 46, (11), 114008.
27. Miller, O. J.; Bernath, K.; Agresti, J. J.; Amitai, G.; Kelly, B. T.; Mastrobattista, E.; Taly, V.; Magdassi, S.; Tawfik, D. S.; Griffiths, A. D., Directed evolution by in vitro compartmentalization. Nature methods 2006, 3, (7), 561-570.
28. Patel, S. K.; Patrick, M. J.; Pollock, J. A.; Janjic, J. M., Two-color fluorescent (near-infrared and visible) triphasic perfluorocarbon nanoemulsions. Journal of biomedical optics 2013, 18, (10), 101312-101312.
29. Quevedo, E.; Steinbacher, J.; McQuade, D. T., Interfacial polymerization within a simplified microfluidic device: capturing capsules. Journal of the American Chemical Society 2005, 127, (30), 10498-10499.
30. Lone, S.; Lee, H. M.; Kim, G. M.; Koh, W.-G.; Cheong, I. W., Facile and highly efficient microencapsulation of a phase change material using tubular microfluidics. Colloids and Surfaces A: Physicochemical and Engineering Aspects 2013, 422, 61-67.
31. Choi, S. W.; Zhang, Y.; Xia, Y., Fabrication of microbeads with a controllable hollow interior and porous wall using a capillary fluidic device. Advanced functional materials 2009, 19, (18), 2943-2949.
32. Choi, S. W.; Cheong, I. W.; Kim, J. H.; Xia, Y., Preparation of Uniform Microspheres Using a Simple Fluidic Device and Their Crystallization into Close-Packed Lattices. Small 2009, 5, (4), 454-459.
33. Arya, C.; Kralj, J. G.; Jiang, K.; Munson, M. S.; Forbes, T. P.; DeVoe, D. L.; Raghavan, S. R.; Forry, S. P., Capturing rare cells from blood using a packed bed of custom-synthesized chitosan microparticles. Journal of Materials Chemistry B 2013, 1, (34), 4313-4319.
34. Davison, P. F.; Freifelder, D., The physical properties of T7 bacteriophage. Journal of molecular biology 1962, 5, (6), 635-IN2.
35. Boitard, L.; Cottinet, D.; Kleinschmitt, C.; Bremond, N.; Baudry, J.; Yvert, G.; Bibette, J., Monitoring single-cell bioenergetics via the coarsening of emulsion droplets. Proceedings of the National Academy of Sciences 2012, 109, (19), 7181-7186.
36. Lorber, N.; Pavageau, B.; Mignard, E., Droplet-based millifluidics as a new miniaturized tool to investigate polymerization reactions. Macromolecules 2010, 43, (13), 5524-5529.
37. Theberge, A. B.; Courtois, F.; Schaerli, Y.; Fischlechner, M.; Abell, C.; Hollfelder, F.; Huck, W. T., Microdroplets in microfluidics: an evolving platform for discoveries in chemistry and biology. Angewandte Chemie International Edition 2010, 49, (34), 5846-5868.
38. Kintses, B.; van Vliet, L. D.; Devenish, S. R.; Hollfelder, F., Microfluidic droplets: new integrated workflows for biological experiments. Current opinion in chemical biology 2010, 14, (5), 548-555.
39. Wu, S.-J. L.; Lee, E. M.; Putvatana, R.; Shurtliff, R. N.; Porter, K. R.; Suharyono, W.; Watts, D. M.; King, C.-C.; Murphy, G. S.; Hayes, C. G., Detection of dengue viral RNA using a nucleic acid sequence-based amplification assay. Journal of clinical microbiology 2001, 39, (8), 2794-2798.
40. Thackray, L. B.; Wobus, C. E.; Chachu, K. A.; Liu, B.; Alegre, E. R.; Henderson, K. S.; Kelley, S. T.; Virgin, H. W., Murine noroviruses comprising a single genogroup exhibit biological diversity despite limited sequence divergence. Journal of virology 2007, 81, (19), 10460-10473.
41. Edgar, R.; McKinstry, M.; Hwang, J.; Oppenheim, A. B.; Fekete, R. A.; Giulian, G.; Merril, C.; Nagashima, K.; Adhya, S., High-sensitivity bacterial detection using biotin-tagged phage and quantum-dot nanocomplexes. Proceedings of the National Academy of Sciences of the U.S. Pat. No. 2,006,103, (13), 4841-4845.
42. Lillehaug, D., An improved plaque assay for poor plaque-producing temperate lactococcal bacteriophages. Journal of applied microbiology 1997, 83, (1), 85-90.
43. Santos, S. B.; Carvalho, C. M.; Sillankorva, S.; Nicolau, A.; Ferreira, E. C.; Azeredo, J., The use of antibiotics to improve phage detection and enumeration by the double-layer agar technique. BMC microbiology 2009, 9, (1), 148.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of assaying for bacteriophage in a sample suspected of comprising bacteriophage, comprising:
a) creating a water-in-oil (W/O) emulsion, comprising:
i) suspending a bacterial cell mixture in an inner aqueous phase (W₁) comprising a water soluble emulsifier and a cell viability dye, wherein the bacterial cell mixture comprises the sample suspected of comprising bacteriophage; and
ii) suspending droplets of the inner aqueous phase (W₁) into an oil phase (O) comprising an oil and a hydrophobic emulsifier having an HLB value of 4 or less, thereby yielding a water-in-oil (W₁/O) emulsion; and
b) detecting the cell viability dye, wherein detectable cell viability dye provides a signal when bacterial cells within the water-in-oil (W₁/O) emulsion are non-viable, thereby indicating the presence of bacteriophage in the sample suspected of comprising bacteriophage.

2. A method of assaying for bacteria strains that are resistant to bacteriophage lysis, comprising:
a) creating a water-in-oil (W/O) emulsion, comprising:
i) suspending a bacterial cell mixture in an inner aqueous phase (W₁) comprising a water soluble emulsifier and a cell viability dye; and
ii) suspending droplets of the inner aqueous phase (W₁) into an oil phase (O) comprising an oil and a hydrophobic emulsifier having an HLB value of 4 or less, thereby yielding a water-in-oil (W₁/O) emulsion; and
b) detecting the cell viability dye, wherein detectable cell viability dye provides a signal when bacterial cells within the water-in-oil (W₁/O) emulsion are non-viable, thereby indicating the presence of bacteria susceptible to bacteriophage in the bacterial cell culture or mixture; and wherein nondetectable cell viability dye indicates the presence of bacteria resistant to bacteriophage in the bacterial cell culture or mixture.

3. A method of assaying for bacteriophage in a sample suspected of comprising bacteriophage, comprising:
a) creating a water-in-oil-in-water (W₁/O/W₂) emulsion, comprising:
i) suspending a bacterial cell mixture in an inner aqueous phase (W₁) comprising a water soluble emulsifier and a cell viability dye, wherein the bacterial cell mixture comprises the sample suspected of comprising bacteriophage;
ii) suspending droplets of the inner aqueous phase (W₁) into an oil phase (O) comprising an oil and a hydrophobic emulsifier having an HLB value of 4 or less, thereby yielding a water-in-oil (W₁/O) emulsion; and
iii) mixing the water-in-oil (W₁/O) emulsion in an outer aqueous phase (W₂), comprising at least one water soluble emulsifier having a hydrophilic lipophilic balance (HLB) value of 7 or greater, thereby creating a water-in-oil-in-water (W₁/O/W₂) emulsion comprising bacterial cells; and
b) detecting the cell viability dye, wherein detectable cell viability dye provides a signal when bacterial cells within the water-in-oil-in-water (W₁/O/W₂) emulsion are non-viable, thereby indicating the presence of bacteriophage in the sample suspected of comprising bacteriophage.

4. A method of assaying for bacteria strains that are resistant to bacteriophage lysis, comprising:
a) creating a water-in-oil-in-water (W₁/O/W₂) emulsion, comprising:
i) suspending a bacterial cell mixture in an inner aqueous phase (W₁) comprising a water soluble emulsifier and a cell viability dye;
ii) suspending droplets of the inner aqueous phase (W₁) into an oil phase (O) comprising an oil and a hydrophobic emulsifier having an HLB value of 4 or less, thereby yielding a water-in-oil (W₁/O) emulsion; and
iii) mixing the water-in-oil (W₁/O) emulsion in an outer aqueous phase (W₂), comprising at least one water soluble emulsifier having a hydrophilic lipophilic balance (HLB) value of 7 or greater, thereby creating a water-in-oil-in-water (W₁/O/W₂) emulsion comprising bacterial cells; and
b) detecting the cell viability dye, wherein detectable cell viability dye provides a signal when bacterial cells within the water-in-oil (W/O) emulsion are non-viable, thereby indicating the presence of bacteria susceptible to bacteriophage in the bacterial cell culture or mixture; and wherein nondetectable cell viability dye indicates the presence of bacteria resistant to bacteriophage in the bacterial cell culture or mixture.

5. The method of any one of claim 1 or 3, further comprising before step a) i) the step of mixing a sample suspected of comprising bacteriophage with a population of bacterial cells, thereby yielding a bacterial cell mixture.

6. The method of claim 5, wherein the sample is a food product.

7. The method of any one of claims 1 to 6, wherein the detecting step comprises performing visual inspection.

8. The method of claim 7, wherein the method detects bacteriophage with a sensitivity of about 10⁴ PFU/mL or less by visual inspection.

9. The method of any one of claims 1 to 8, wherein the detecting step comprises performing optical microscopy

10. The method of any one of claims 1 to 8, wherein the detecting step comprises performing flow cytometry.

11. The method of any one of claims 9 to 10, wherein the method detects bacteriophage with a sensitivity of about 10² PFU/mL or less by optical microscopy or flow cytometry.

12. The method of any one of claims 1 to 11, wherein the detecting step does not comprise performing one or more of flow cytometry, impedance spectroscopy or nucleic acid amplification.

13. The method of any one of claims 1 to 12, wherein the method can be performed in 2 or fewer hours.

14. The method of any one of claims 1 to 13, wherein the hydrophilic emulsifier in the inner aqueous phase (W₁) has a hydrophilic lipophilic balance (HLB) value of 10 or greater.

15. The method of claim 14, wherein the hydrophilic lipophilic balance (HLB) value of 10 or greater is a protein-based or proteinaceous emulsifier.

16. The method of any one of claims 1 to 13, wherein the hydrophilic emulsifier in the inner aqueous phase (W₁) comprises a particle-based emulsifier.

17. The method of any one of claims 1 to 16, wherein the cell viability dye is a fluorophore.

18. The method of any one of claims 1 to 17, wherein the cell viability dye binds to or intercalates into DNA.

19. The method of claim 18, wherein the cell viability dye is selected from the group consisting of propidium iodide (PI), 7-aminoactinomycin D (7-AAD), DRAQ7™, and TO-PRO®-3 Iodide.

20. The method of any one of claims 1 to 17, wherein the cell viability dye is selected from propidium iodide (PI), hexidium iodide, a carbocyanine, rhodamine 123, tetra methyl rhodamine, dialkylaminophenylpolyenylpyridinium, aminonaphthylethenylpyridinium, resazurin, formazan, red-fluorescent ethidium homodimer-1, calcein, tetrasodium (6E,6′E)-6,6-[(3,3′-dimethylbiphenyl-4,4′-diyl)di(1E)hydrazin-2-yl-1-ylidene]bis(4-amino-5-oxo-5,6-dihydronaphthalene-1,3-disulfonate) (Evans blue), (3Z,3′Z)-3,3′-[(3,3′-dimethylbiphenyl-4,4′-diyl)di(1Z)hydrazin-2-yl-1-ylidene]bis(5-amino-4-oxo-3,4-dihydronaphthalene-2,7-disulfonic acid) (Trypan blue), 7 aminoactinomycin D (7-AAD), DRAQ7™, eFluor® 455UV, eFluor® 450, eFluor® 506, eFluor® 520, eFluor® 660, eFluor® 780, Zombie Aqua™, Zombie Green™, Zombie NIR™, Zombie Red™, Zombie Violet™, Zombie UV™, and Zombie Yellow™.

21. The method of any one of claims 1 to 13, wherein the cell viability dye is a colorimetric dye.

22. The method of any one of claims 1 to 21, wherein the one or more bacteriophages are lytic bacteriophages.

23. The method of any one of claims 1 to 21, wherein the one or more bacteriophages are lysogenic or temperate bacteriophages.

24. The method of claim 23, further comprising prior to the detecting stepm inducing the lytic cycle of the lysogenic or temperate bacteriophages.

25. The method of any one of claims 1 to 23, wherein the one or more bacteriophages are a member of a viral family selected from the group consisting of Myoviridae, Siphoviridae, Podoviridae, Lipothrixviridae, Rudiviridae, Ampullaviridae, Bicaudaviridae, Clavaviridae, Corticoviridae, Cystoviridae, Fuselloviridae, Globuloviridae, Guttavirus, Inoviridae, Leviviridae, Microviridae, Plasmaviridae, and Tectiviridae.

26. The method of any one of claims 1 to 25, wherein the one or more bacteriophages are lytic to a bacterial cell selected from the group consisting of Campylobacter, Cronobacter, Escherichia, Salmonella, Lactococcus, Vibrio, Erwinia, Xanthomonas, Shigella, Staphylococcus, Streptococcus, Clostridium, Pseudomonas, Mycobacterium, Neisseria, and Bacilli.

27. The method of any one of claims 1 to 26, wherein the bacteriophages are selected from the group consisting of lactococcal phage species (936, c2, c6A, 1483, T187, P087, 1358, KSY1, 949, and P335 phage species), T4 phage, T7 phage, phage A1511, phage Felix-O1, phage PHL 4, phage P7, ECML-4, ECML-117, ECML-134, phage A511, phage P100, ATCC accession no. PTA-5372, ATCC accession no. PTA-5373, ATCC accession no. PTA-5374, ATCC accession no. PTA-5375, ATCC accession no. PTA-5376, ATCC accession no. PTA-5377, phage FO1-E2, phage CJ6, phage Φ88, phage Φ35, NgoΦ6 and NgoΦ7, lambdoid prophages, phage β, Lambda phages, Mu-1, lactococcal lysogenic phages (φLC3, Tuc2009, bIL285, bIL286 and bIL309, biL170, bL167), Lysogenic phages of S. aureus (8325-4, Ps6, 655, 248, W-26, U9, 655C, Oh-SO, 608, N-135, C-72), and mixtures thereof.

28. The method of any one of claims 1 to 27, wherein the oil in the oil phase is liquid at 25-30° C.

29. The method of any one of claims 1 to 28, wherein the oil in the oil phase is selected from mineral oil, canola oil, olive oil, corn oil, sunflower oil, safflower oil, peanut oil, coconut oil and perfluorodecalin.

30. The method of any one of claims 1 to 29, wherein the hydrophobic emulsifier comprises a polyglycerol ester of fatty acid.

31. The method of any one of claims 1 to 30, wherein the hydrophobic emulsifier comprises polyglycerol polyricinoleate (PGPR).

32. The method of any one of claims 3 to 31, wherein the outer aqueous phase (W2) emulsifier having a hydrophilic lipophilic balance (HLB) value of 7 or greater comprises a mixture comprising a bile salt, a zwitterionic detergent and a nonionic detergent.

33. The method of any one of claims 3 to 32, wherein the outer aqueous phase (W2) comprises one or more bile salts, lecithin and Tween 20.

34. The method of any one of claims 1 to 33, wherein the detecting step is performed in comparison to a control comprising the inner aqueous phase without bacteriophage.

35. A portable device comprising a tubing in fluid communication from the upstream to downstream direction, with (i) a fluidic droplet generator, (ii) an incubator and (iii) a detector, wherein:
a) the upstream end of the tubing is in fluid communication with a sample reservoir;
b) the tubing within the fluidic droplet generator comprises a first upstream syringe comprising a needle comprising a beveled tip, wherein the beveled tip is pierced into the inner space of the tubing, wherein the inner space of the first syringe comprises an oil phase comprising an emulsifier; and a second downstream syringe comprising a needle comprising a beveled tip, wherein the beveled tip is pierced into the inner space of the tubing downstream from the needle of the first syringe, wherein the inner space of the second syringe comprises an aqueous phase comprising at least one detergent;
c) the incubator can hold a preselected or predetermined temperature in the range of about 4° C. to about 50° C.; and
d) the detector can detect a fluorescent or colorimetric signal.

36. The portable device of claim 35, wherein one or more of the sample reservoir, the first syringe and the second syringe automatically deliver fluid.

37. The portable device of any one of claims 35 to 36, wherein the device weighs less than 10 kg.

38. The portable device of any one of claims 35 to 37, wherein the device has a desk or table footprint of less than about 200 in².

39. The portable device of any one of claims 35 to 38, wherein the inner space of the tubing has a diameter in the range of about 1/32 (0.03125) inches to about 1/16 (0.0625) inches.

40. The portable device of any one of claims 35 to 39, wherein the needle of the first syringe and/or the second syringe has a gauge from about 25 G to about 30 G.

41. The portable device of any one of claims 35 to 40, wherein the device is as depicted in FIGS. 8, 9 and/or 10.

42. A microfluidic device for creating water-in-oil-in-water (W₁/O/W₂) emulsion droplets, comprising:
i) a first inlet in fluid communication with a first lumen, the first inlet and first lumen comprising an inner aqueous phase;
ii) a second inlet in fluid communication with a second lumen, the second inlet and second lumen comprising an oil phase, wherein the second lumen is in fluid communication with the first lumen;
iii) a third inlet in fluid communication with a third lumen, the third inlet and third lumen comprising an outer aqueous phase, wherein the third lumen is in fluid communication with the first lumen, wherein the third lumen connects with the first lumen downstream of where the second lumen connects with the first lumen; and
iv) an outlet for collecting water-in-oil-in-water (W₁/O/W₂) emulsion droplets, wherein the outlet is in fluid communication with the first lumen.

43. A microfluidic device for creating water-in-oil (W₁/O) emulsion droplets, comprising:
i) a first inlet in fluid communication with a first lumen, the first inlet and first lumen comprising an inner aqueous phase;
ii) a second inlet in fluid communication with a second lumen, the second inlet and second lumen comprising an oil phase, wherein the second lumen is in fluid communication with the first lumen; and
iii) an outlet for collecting water-in-oil (W₁/O) emulsion droplets, wherein the outlet is in fluid communication with the first lumen.

44. The microfluidic device of any one of claims 42 to 43, wherein the inner diameters of the first, second and third lumens are from about 30 μm to about 150 μm.

45. The microfluidic device of any one of claims 42 to 44, wherein the device is as depicted in FIGS. 11-12.
